(19) United States
(12) Patent Application Publication
(10) Pub. No.: US 2026/0248777 A1
(43) Pub. Date: Aug. 27, 2026

(54) METHODS OF TREATING CANCER

(71) Applicant: FOGHORN THERAPEUTICS INC., Cambridge, MA (US)

(72) Inventors: Michael P. COLLINS, Medford, MA (US); Jessica C. PIEL, Natick, MA (US); Jessica Y. WAN, Newton, MA (US)

(21) Appl. No.: 18/879,368

(22) PCT Filed: Jun. 27, 2023
(86) PCT No.: PCT/US2023/026363

§ 371 Date: Dec. 27, 2024

Related U.S. Application Data
(60) Provisional application No. 63/355,870, filed on Jun. 27, 2022.
(60) Provisional application No. 63/482,718, filed on Feb. 1, 2023.

(51) Int. Cl. A61K 31/4545; A61P 35/00

(52) U.S. Cl. CPC A61K 31/4545; A61P 35/00

(57) ABSTRACT

The present disclosure relates to methods and compositions for the treatment of cancer.

### BACKGROUND

Disorders can be affected by the BAF complex. Bromodomain-containing protein 9 (BRD9), a component of the non-canonical BAF (BRG1- or BRM-associated factors) complex, a SWI/SNF ATPase chromatin remodeling complex, and belongs to family IV of the bromodomain-containing proteins. BRD9 is a protein encoded by the BRD9 gene on chromosome 5 and is present in several SWI/SNF ATPase chromatin remodeling complexes.

BRD9 is upregulated in multiple cancer cell lines. Accordingly, agents that reduce the levels and/or activity of BRD9 may provide new methods for the treatment of disease and disorders, such as cancer. It was found that depleting BRD9 in cells results in the depletion of the SS18-SSX fusion protein in those cells. The SS18-SSX fusion protein has been detected in more than 95% of synovial sarcoma tumors and is often the only cytogenetic abnormality in synovial sarcoma. Additionally, evidence suggests that the non-canonical BAF complex is involved in cellular antiviral activities. Thus, agents that degrade BRD9 (e.g., compounds) are useful in the treatment of disorders (e.g., cancers or infections) related to non-canonical BAF, BRD9, and/or SS18-SSX.

There is a continual need to provide novel methods for the treatment of non-canonical BAF complex-related disorders, such as cancer.

### SUMMARY

The present disclosure features compounds and methods useful for treating non-canonical BAF-related disorders (e.g., cancer).

In one aspect, the present disclosure provides a method of treating cancer in a subject in need thereof. The method includes administering an effective amount of a compound that reduces the level and/or activity of BRD9, or a pharmaceutically acceptable salt thereof, in which the effective amount is an amount sufficient to decrease a BRD9 immunohistochemistry score of the subject.

In another aspect, the present disclosure provides a method of decreasing a BRD9 immunohistochemistry score in a subject. The method incudes administering an effective amount of a compound that reduces the level and/or activity of BRD9, or a pharmaceutically acceptable salt thereof.

In some embodiments of any of the aspects disclosed herein, the effective amount is an amount sufficient to decrease a BRD9 immunohistochemistry score in the subject compared to a BRD9 immunohistochemistry score of the subject prior to the administration of the compound or a pharmaceutically acceptable salt thereof.

In some embodiments of any of the aspects disclosed herein, the effective amount is an amount sufficient to decrease a BRD9 immunohistochemistry score in the subject compared to a reference BRD9 immunohistochemistry score.

In some embodiments of any of the aspects disclosed herein, the effective amount is an amount sufficient to decrease a BRD9 immunohistochemistry score in the subject compared to a BRD9 immunohistochemistry score of a subject who was not administered the compound or a pharmaceutically acceptable salt thereof.

In some embodiments of any of the aspects disclosed herein, the BRD9 immunohistochemistry score is an H score.

In some embodiments of any of the aspects disclosed herein, the effective amount is an amount sufficient to reduce a BRD9 immunohistochemistry score to a score of between about 15 and about 0.

In some embodiments of any of the aspects disclosed herein, the BRD9 immunohistochemistry score is determined for a tumor in the subject.

In some embodiments of any of the aspects disclosed herein, the effective amount is an amount sufficient to decrease a BRD9 immunohistochemistry score of a tumor in the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the BRD9 immunohistochemistry score in the tumor of the subject prior to administration of the compound or a pharmaceutically acceptable salt thereof. In some embodiments of any of the aspects disclosed herein, the effective amount is an amount sufficient to decrease a BRD9 immunohistochemistry score of a tumor in the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the BRD9 immunohistochemistry score in the tumor of the subject prior to administration of the compound or a pharmaceutically acceptable salt thereof.

In some embodiments of any of the aspects described herein, the effective amount is an amount sufficient to maintain a BRD9 immunohistochemistry score that is between about 15 and about 0 in the subject, over at least 14 days.

In some embodiments of any of the aspects described herein, the effective amount is an amount sufficient to maintain a BRD9 immunohistochemistry score that is between about 15 and about 0 in the subject, over at least 21 days.

In some embodiments of any of the aspects described herein, the effective amount is an amount sufficient to maintain a BRD9 immunohistochemistry score that is between about 15 and about 0 in the subject, over at least 28 days.

In another aspect, the present disclosure provides a method of treating cancer in a subject in need thereof. The method includes administering an effective amount of a compound that reduces the level and/or activity of BRD9, or a pharmaceutically acceptable salt thereof, wherein the effective amount is an amount sufficient to reduce the level of BRD9 expression in the subject.

In another aspect, the method of maintaining a reduction in levels of BRD9 expression in a subject, the method comprising administering an effective amount of a compound that reduces the level and/or activity of BRD9, or a pharmaceutically acceptable salt thereof.

In some embodiments of any of the aspects disclosed herein, the effective amount is an amount sufficient to reduce the level of BRD9 expression in the subject compared to the level of BRD9 expression in the subject prior to the administration of the compound or a pharmaceutically acceptable salt thereof.

In some embodiments of any of the aspects described herein, the effective amount is an amount sufficient to reduce the level of BRD9 expression in the subject compared to the level of BRD9 expression of a reference.

In some embodiments of any of the aspects described herein, the effective amount is an amount sufficient to reduce the level of BRD9 expression in a subject compared to the level of BRD9 expression in a subject who was not administered the compound or a pharmaceutically acceptable salt thereof.

In some embodiments of any of the aspects described herein, the levels of BRD9 expression are determined in a blood sample of the subject.

In some embodiments of any of the aspects described herein, the effective amount is an amount sufficient to reduce the level of BRD9 expression in a blood sample of the subject by between about 90% and about 100% compared to the level of BRD9 expression in a blood sample of the subject prior to administration of the compound or a pharmaceutically acceptable salt thereof.

In some embodiments of any of the aspects described herein, the levels of BRD9 expression are determined in a tumor of the subject.

In some embodiments of any of the aspects described herein, the effective amount is an amount sufficient to reduce the level of BRD9 expression in a tumor of the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the level of BRD9 expression in the tumor of the subject prior to administration of the compound or a pharmaceutically acceptable salt thereof. In some embodiments of any of the aspects described herein, the effective amount is an amount sufficient to reduce the level of BRD9 expression in a tumor of the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the level of BRD9 expression in the tumor of the subject prior to administration of the compound or a pharmaceutically acceptable salt thereof.

In some embodiments of any of the aspects described herein, the effective amount is an amount sufficient to maintain a reduction of BRD9 expression of between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) in the subject, over at least 14 days.

In some embodiments of any of the aspects described herein, the effective amount is an amount sufficient to maintain a reduction of BRD9 expression by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) in the subject, over at least 14 days.

In some embodiments of any of the aspects described herein, the effective amount is an amount sufficient to maintain a reduction of BRD9 expression of between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) in the subject, over at least 21 days.

In some embodiments of any of the aspects described herein, the effective amount is an amount sufficient to maintain a reduction of BRD9 expression by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) in the subject, over at least 21 days.

In some embodiments of any of the aspects described herein, the effective amount is an amount sufficient to maintain a reduction of BRD9 expression of between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) in the subject, over at least 28 days.

In some embodiments of any of the aspects described herein, the effective amount is an amount sufficient to maintain a reduction of BRD9 expression by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) in the subject, over at least 28 days.

In some embodiments of any of the aspects described herein, the effective amount is an amount sufficient to cause a greater than or equal to 10% decrease in tumor size and/or a greater than or equal to 15% decrease in tumor attenuation in the subject. In some embodiments of any of the aspects described herein, the subject has no new lesions. In some embodiments of any of the aspects described herein, the subject has no obvious progression of non-measurable disease.

In some embodiments of any of the aspects described herein, the effective amount is an amount sufficient to maintain a plasma concentration of 3 ng/mL of the compound in a subject, over at least 14 days.

In some embodiments of any of the aspects described herein, the effective amount is an amount sufficient to maintain a plasma concentration of 3 ng/mL of the compound in a subject, over at least 21 days.

In some embodiments of any of the aspects described herein, the effective amount is an amount sufficient to maintain a plasma concentration of 3 ng/mL of the compound in a subject, over at least 28 days.

In one aspect, the present disclosure provides a method of treating cancer in a subject in need thereof. The method includes administering an effective amount of a compound that reduces the level and/or activity of BRD9, or a pharmaceutically acceptable salt thereof, in which the effective amount is an amount sufficient to decrease a BRD9 immunohistochemistry score of the subject and/or decrease Ki67 protein levels in the subject.

In another aspect, the present disclosure provides a method of decreasing Ki67 protein levels in a subject. The method incudes administering an effective amount of a compound that reduces the level and/or activity of BRD9, or a pharmaceutically acceptable salt thereof.

In some embodiments of any of the aspects disclosed herein, the effective amount is an amount sufficient to decrease a Ki67 protein levels in the subject compared to Ki67 protein levels of the subject prior to the administration of the compound or a pharmaceutically acceptable salt thereof.

In some embodiments of any of the aspects disclosed herein, the effective amount is an amount sufficient to decrease Ki67 protein levels in the subject compared to a reference Ki67 protein level.

In some embodiments of any of the aspects disclosed herein, the effective amount is an amount sufficient to decrease Ki67 protein levels in the subject compared to Ki67 protein levels of a subject who was not administered the compound or a pharmaceutically acceptable salt thereof.

In some embodiments of any of the aspects disclosed herein, the effective amount is an amount sufficient to reduce Ki67 protein levels to between about 50% and about 0%.

In some embodiments of any of the aspects disclosed herein, Ki67 protein levels are determined for a tumor in the subject.

In some embodiments of any of the aspects disclosed herein, the effective amount is an amount sufficient to reduce Ki67 protein levels of a tumor in the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the Ki67 protein levels in the tumor of the subject prior to administration of the compound or a pharmaceutically acceptable salt thereof. In some embodiments of any of the aspects disclosed herein, the effective amount is an amount sufficient to reduce Ki67 protein levels of a tumor in the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the Ki67 protein levels in the tumor of the subject prior to administration of the compound or a pharmaceutically acceptable salt thereof.

In some embodiments of any of the aspects described herein, the effective amount is an amount sufficient to maintain a reduction of Ki67 protein levels of between about 35% and about 80% in the subject, over at least 14 days.

In some embodiments of any of the aspects described herein, the effective amount is an amount sufficient to maintain a reduction of Ki67 protein levels of between about 35% and about 80% in the subject, over at least 21 days.

In some embodiments of any of the aspects described herein, the effective amount is an amount sufficient to maintain a reduction of Ki67 protein levels of between about 35% and about 80% in the subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to maintain a plasma concentration of 3 ng/mL of the compound in a subject.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size or a greater than or equal to 15% decrease in tumor attenuation.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and a greater than or equal to 15% decrease in tumor attenuation.

In some embodiments of any of the aspects described herein, the subject has no new lesions.

In some embodiments of any of the aspects described herein, the subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation, no progression of non-target lesions, and no new lesions.

In some embodiments of any of the aspects described herein, the subject is evaluated for disease progression using Choi criteria.

In some embodiments of any of the aspects described herein, the subject is evaluated for disease progression using mChoi criteria.

In some embodiments of any of the aspects described herein, the subject has a partial response to cancer therapy.

In some embodiments of any of the aspects described herein, tumor size and/or tumor attenuation of the subject is measured by computed tomography (CT).

In some embodiments of any of the aspects described herein, the cancer is a malignant rhabdoid tumor, a CDS+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma (e.g., e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, or low-grade rhabdomyosarcoma), non-small cell lung cancer, stomach cancer, breast cancer, or SMARCAB1-loss cancer (e.g., high grade ovarian serous adenocarcinoma, epithelioid sarcoma, atypical teratoid/rhabdoid tumor, prostate adenocarcinoma, conventional glioblastoma multiforme, breast invasive ductal carcinoma, sarcoma (NOS), gastrointestinal stromal tumor, kidney medullary carcinoma, pancreatic neuroendocrine neoplasm, sinonasal adenocarcinoma, adrenal cortex carcinoma, proximal-type epithelioid sarcoma, head and neck carcinoma, invasive breast carcinoma, rhabdoid tumor of the kidney, dedifferentiated liposarcoma, pancreatic adenocarcinoma, chordoma, testicular yolk sac tumor, small cell lung carcinoma, squamous cell lung carcinoma, acral lentiginous melanoma, peripheral T-cell lymphoma, nasal cavity and paranasal sinus squamous cell carcinoma, malignant peripheral nerve sheath tumor, breast invasive lobular carcinoma, chordoma, a SMARCB1-mutant cancer, or lung adenocarcinoma).

In some embodiments of any of the aspects described herein, the compound is 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione, or a pharmaceutically acceptable salt thereof.

In some embodiments of any of the aspects described herein, the compound is:

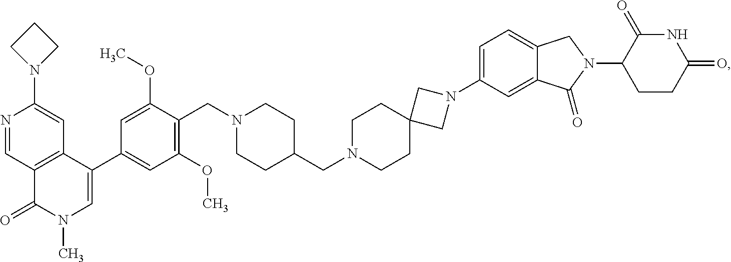 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

or a pharmaceutically acceptable salt thereof.

In some embodiments of any of the aspects described herein, the compound is:

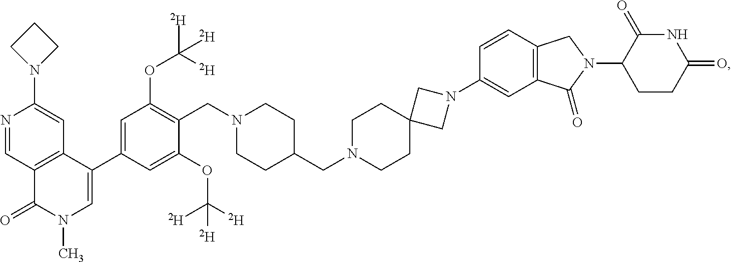 [SMILES: [2H]C([2H])([2H])Oc1cc(-c2cn(C)c(=O)c3cnc(N4CCC4)cc23)cc(OC([2H])([2H])[2H])c1CN1CCC(CN2CCC3(CC2)CN(c2ccc4c(c2)C(=O)N(C2CCC(=O)NC2=O)C4)C3)CC1]

or a pharmaceutically acceptable salt thereof.

In some preferred embodiments of any of the aspects described herein, the compound is compound A:

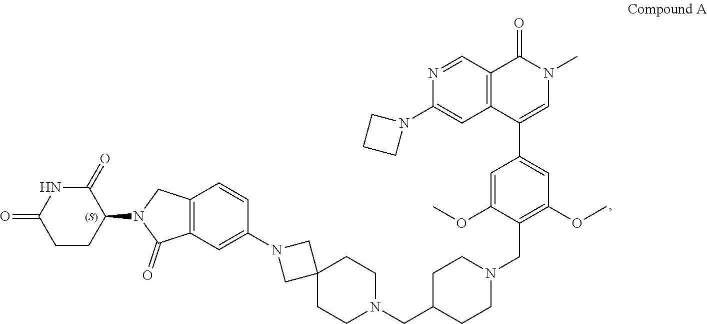

or a pharmaceutically acceptable salt thereof.

In some embodiments of any of the aspects described herein, the compound is:

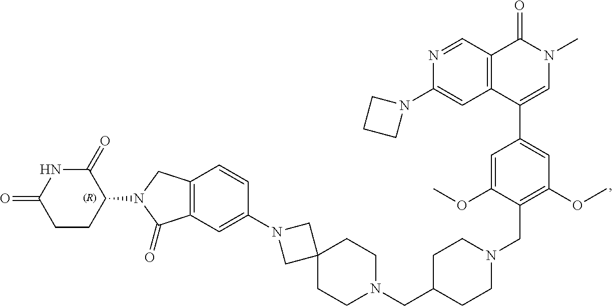

or a pharmaceutically acceptable salt thereof.

In some embodiments of any of the aspects disclosed herein, the effective amount of the compound or a pharmaceutically acceptable salt thereof, is administered in a dose of 20-40 mg/kg (e.g., 20-60 mg/kg, 20-40 mg/kg, 40-80 mg/kg, 40-60 mg/kg, or 60-80 mg/kg).

In some embodiments of any of the aspects disclosed herein, the effective amount of the compound or a pharmaceutically acceptable salt thereof, is administered in a dose of 20 mg/kg. In some embodiments of any of the aspects disclosed herein, the effective amount of the compound or a pharmaceutically acceptable salt thereof, is administered in a dose of 40 mg/kg. In some embodiments of any of the aspects disclosed herein, the effective amount of the compound or a pharmaceutically acceptable salt thereof, is administered in a dose of 60 mg/kg. In some embodiments of any of the aspects disclosed herein, the effective amount of the compound or a pharmaceutically acceptable salt thereof, is administered in a dose of 80 mg/kg.

In some embodiments of any of the aspects disclosed herein, the effective amount of the compound or a pharmaceutically acceptable salt thereof, is administered to the subject at least once per week (e.g., once per week).

In some embodiments of any of the aspects disclosed herein, the effective amount of the compound or a pharmaceutically acceptable salt thereof, is administered to the subject at least twice per week (e.g., twice per week).

In some embodiments of any of the aspects disclosed herein, the effective amount of the compound or a pharmaceutically acceptable salt thereof, is administered to the subject at least once per week.

In some embodiments of any of the aspects disclosed herein, the effective amount of the compound or a pharmaceutically acceptable salt thereof, is administered to the subject at least twice per week.

In some embodiments of any of the aspects disclosed herein, the effective amount of the compound or a pharmaceutically acceptable salt thereof, is administered in a dose of 20 mg/kg once per week. In some embodiments of any of the aspects disclosed herein, the effective amount of the compound or a pharmaceutically acceptable salt thereof, is administered in a dose of 20 mg/kg twice per week.

In some embodiments of any of the aspects disclosed herein, the effective amount of the compound or a pharmaceutically acceptable salt thereof, is administered in a dose of 40 mg/kg once per week. In some embodiments of any of the aspects disclosed herein, the effective amount of the compound or a pharmaceutically acceptable salt thereof, is administered in a dose of 40 mg/kg twice per week.

In some embodiments of any of the aspects disclosed herein, the effective amount of the compound or a pharmaceutically acceptable salt thereof, is administered in a dose of 60 mg/kg once per week. In some embodiments of any of the aspects disclosed herein, the effective amount of the compound or a pharmaceutically acceptable salt thereof, is administered in a dose of 60 mg/kg twice per week.

In some embodiments of any of the aspects disclosed herein, the effective amount of the compound or a pharmaceutically acceptable salt thereof, is administered in a dose of 80 mg/kg once per week. In some embodiments of any of the aspects disclosed herein, the effective amount of the compound or a pharmaceutically acceptable salt thereof, is administered in a dose of 80 mg/kg twice per week.

In some embodiments of any of the aspects disclosed herein, the compound or a pharmaceutically acceptable salt thereof is administered to the subject in a 14-day dosing cycle.

In some embodiments of any of the aspects disclosed herein, the compound or a pharmaceutically acceptable salt thereof is administered to the subject in a 21-day dosing cycle.

In some embodiments of any of the aspects disclosed herein, the compound or a pharmaceutically acceptable salt thereof is administered to the subject in a 28-day dosing cycle.

In some embodiments of any of the aspects disclosed herein, the compound or a pharmaceutically acceptable salt thereof, is administered to the subject intravenously.

In some embodiments of any of the aspects disclosed herein, the compound or a pharmaceutically acceptable salt thereof, is administered to the subject subcutaneously.

In some embodiments of any of the aspects disclosed herein, the compound or a pharmaceutically acceptable salt thereof, is administered to the subject intramuscularly.

### Chemical Terms

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “adult soft tissue sarcoma” refers to a sarcoma that develops in the soft tissues of the body, typically in adolescent and adult subjects (e.g., subjects who are at least 10 years old, 11 years old, 12 years old, 13 years old, 14 years old, 15 years old, 16 years old, 17 years old, 18 years old, or 19 years old). Non-limiting examples of adult soft tissue sarcoma include, but are not limited to, synovial sarcoma, fibrosarcoma, malignant fibrous histiocytoma, dermatofibrosarcoma, liposarcoma, leiomyosarcoma, hemangiosarcoma, Kaposi's sarcoma, lymphangiosarcoma, malignant peripheral nerve sheath tumor/neurofibrosarcoma, extraskeletal chondrosarcoma, extraskeletal osteosarcoma, extraskeletal myxoid chondrosarcoma, and extraskeletal mesenchymal chondrosarcoma.

As used herein, the term “BAF complex” refers to the BRG1- or HRBM-associated factors complex in a human cell.

As used herein, the term “BAF complex-related disorder” refers to a disorder that is caused or affected by the level and/or activity of a BAF complex.

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18.

As used herein, the term “BRD9” refers to bromodomain-containing protein 9, a component of the BAF (BRG1- or BRM-associated factors) complex, a SWI/SNF ATPase chromatin remodeling complex, and belongs to family IV of the bromodomain-containing proteins. BRD9 is encoded by the BRD9 gene, the nucleic acid sequence corresponding to positions 863735-892803 in RefSeq sequence NC_000005.10 of GRCh38.p13 (RefSeq assembly accession No. GCF_000001405.39). The term “BRD9” also refers to natural variants of the wild-type BRD9 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BRD9, which is set forth in SEQ ID NO: 1:

MGSSHHHHHHENLYFQGDYKDDDDKGSLEVLFQGPAENESTPIQQLLEHFLRQLQRKDPHGFFAFPVT
DAIAPGYSMIIKHPMDFGTMKDKIVANEYKSVTEFKADFKLMCDNAMTYNRPDTVYYKLAKKILHAGFKM
MSK.

As used herein, the term “BRD9-related disorder” refers to a disorder that is caused or affected by the level and/or activity of BRD9. The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

A “compound of the present disclosure” and similar terms as used herein, whether explicitly noted or not, refers to compounds useful for treating BAF-related disorders (e.g., cancer) described herein, including, e.g., 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione and the compounds disclosed in International Patent Publication Nos. WO/2021/155225, WO/2021/022163, WO/2020/160198, WO/2020/160196, WO/2020/160193, WO/2020/160192, and WO/2019/152440 (the contents of which are incorporated herein by reference in their entirety), as well as salts (e.g., pharmaceutically acceptable salts), solvates, hydrates, stereoisomers (including atropisomers), and tautomers thereof. Those skilled in the art will appreciate that certain compounds described herein can exist in one or more different isomeric (e.g., stereoisomers, geometric isomers, atropisomers, and tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination. Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

As used herein, the term “degrader” refers to a small molecule compound including a degradation moiety, wherein the compound interacts with a protein (e.g., BRD9) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRD9. In one example, the moiety binds to a protease or a ubiquitin ligase that metabolizes the protein, e.g., BRD9.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

As used herein, the terms “effective amount,” “therapeutically effective amount,” and “a “sufficient amount” of an agent that reduces the level and/or activity of BRD9 (e.g., in a cell or a subject) described herein, unless specified otherwise, refer to a quantity sufficient to, when administered to the subject, including a human, decrease a BRD9 immunohistochemistry score of the subject or an amount sufficient to reduce the level of BRD9 expression in the subject, as described herein. An “effective amount” may also refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BRD9 sufficient to achieve a treatment response as compared to the response obtained without administration of the agent that reduces the level and/or activity of BRD9. The amount of a given agent that reduces the level and/or activity of BRD9 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BRD9 of the present disclosure is an amount which results in a beneficial or desired result in a subject, e.g., as compared to a control. As defined herein, a therapeutically effective amount of an agent that reduces the level and/or activity of BRD9 of the present disclosure may be readily determined by one of ordinary skill by routine methods known in the art. Dosage regimen may be adjusted to provide the optimum therapeutic response.

As used herein, the term “inhibitor” refers to any agent which reduces the level and/or activity of a protein (e.g., BRD9). Non-limiting examples of inhibitors include small molecule inhibitors, degraders, antibodies, enzymes, or polynucleotides (e.g., siRNA).

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, “metastatic nodule” refers to an aggregation of tumor cells in the body at a site other than the site of the original tumor.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

As used herein, the term “non-canonical BAF complex” refers to the BRG1- or HRBM-associated factors complex in a human cell that includes BRD9.

As used herein, the term “non-canonical BAF complex-related disorder” refers to a disorder that is caused or affected by the level and/or activity of a non-canonical BAF complex.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid. The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

By “reducing the activity of BRD9,” is meant decreasing the level of an activity related to an BRD9, or a related downstream effect. A non-limiting example of inhibition of an activity of BRD9 is decreasing the level of a BAF complex (e.g., GBAF) in a cell. The activity level of BRD9 may be measured using any method known in the art. In some embodiments, an agent which reduces the activity of BRD9 is a small molecule BRD9 inhibitor. In some embodiments, an agent which reduces the activity of BRD9 is a small molecule BRD9 degrader.

By “reducing the level of BRD9,” is meant decreasing the level of BRD9 in a cell or subject. The level of BRD9 may be measured using any method known in the art.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a compound of the disclosure may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIGS. 1A-1D are graphs illustrating BRD9 protein levels detected by flow cytometry in PBMC subpopulations from 4 subjects treated with 5 mg Compound A. (A) is subject 1, (B) is subject 2, (C) is subject 3, (D) is subject 4.

FIGS. 2A-2D are graphs illustrating BRD9 protein levels detected by flow cytometry in PBMC subpopulations from 4 subjects treated with 10 mg Compound A. (A) is subject 5, (B) is subject 6, (C) is subject 7, (D) is subject 8.

FIGS. 3A-3D show representative images of BRD9 detected by IHC in synovial sarcoma patient tumor biopsies collected at screening and on-treatment visits. (A) is subject 1 screening (Panel 1) and EOT (Panel 2), (B) is subject 2 screening (Panel 1) and C3D1 (Panel 2), (C) is subject 3 screening (Panel 1) and C3D1 (Panel 2), (D) is subject 8 screening (Panel 1) and EOT (Panel 2).

FIG. 4 is a graph illustrating BRD9 levels detected by IHC in SYO-1 xenografts from mice treated with Compound A. Symbols represent group averages, and error bars represent SEM.

FIG. 5 is a graph illustrating mean plasma concentration of Compound A overtime after administration of various nanosuspension and microsuspension formulations tested in a rat pharmacokinetics study. 3 ng/mL is denoted by a black dashed line.

FIG. 6 is a graph illustrating mean plasma concentration of Compound A overtime after administration of various nanosuspension formulations tested in a mouse pharmacokinetics study. 3 ng/mL is denoted by a black dashed line.

FIG. 7A is a graph illustrating body weight of mice after administration of various nanosuspension formulations.

FIG. 7B is a graph illustrating tumor volume of tumors in mice after administration of various nanosuspension formulations.

FIGS. 8A-8C show representative morphology changes observed in H&E images of synovial sarcoma patient tumor biopsies collected at on-treatment visits.

### DETAILED DESCRIPTION

The present disclosure features compositions and methods useful for the treatment of BAF-related disorders (e.g., cancer). The disclosure further features compositions and methods useful for inhibition of the level and/or activity of BRD9, e.g., for the treatment of disorders such as cancer (e.g., sarcoma or a SMARCAB1-loss cancer) in a subject in need thereof.

### Compounds

The present disclosure provides compounds that reduce the level of an activity related to BRD9, or a related downstream effect, or reduce the level of BRD9 in a cell or subject.

In some embodiments of any of the aspects described herein, the compound is a compound described in International Publication No. WO/2021/155225, or a pharmaceutically acceptable salt thereof.

In some embodiments, the compound is 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione:

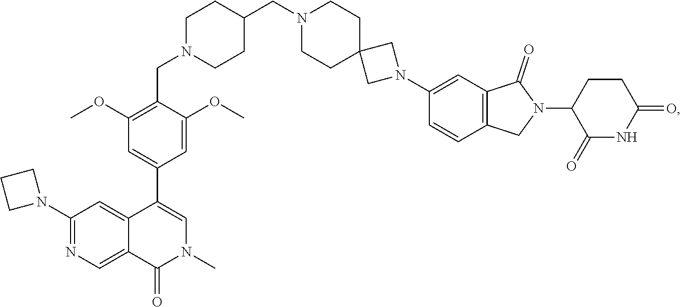 [SMILES: COc1cc(-c2cn(C)c(=O)c3cnc(N4CCC4)cc23)cc(OC)c1CN1CCC(CN2CCC3(CC2)CN(c2ccc4c(c2)C(=O)N(C2CCC(=O)NC2=O)C4)C3)CC1]

or a pharmaceutically acceptable salt thereof.

In some embodiments, the compound is

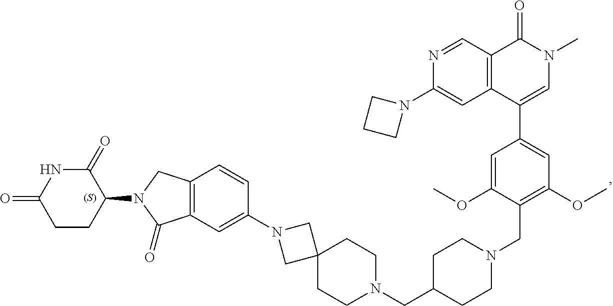

or a pharmaceutically acceptable salt thereof.

In some embodiments, the compound is

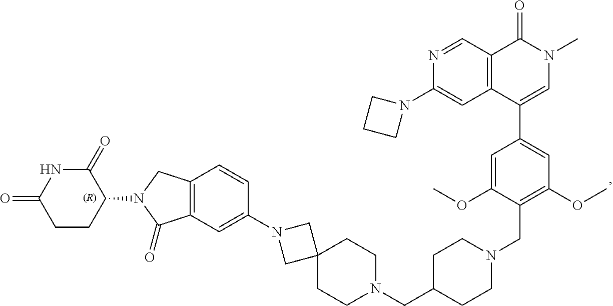

or a pharmaceutically acceptable salt thereof.

In some embodiments, the compound is:

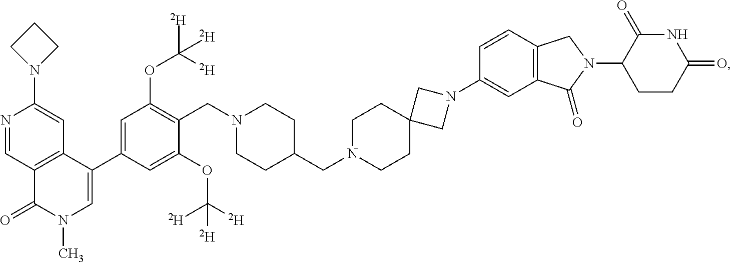 [SMILES: [2H]C([2H])([2H])Oc1cc(-c2cn(C)c(=O)c3cnc(N4CCC4)cc23)cc(OC([2H])([2H])[2H])c1CN1CCC(CN2CCC3(CC2)CN(c2ccc4c(c2)C(=O)N(C2CCC(=O)NC2=O)C4)C3)CC1]

or a pharmaceutically acceptable salt thereof.

In some embodiments of any of the aspects described herein, the compound is a compound described in International Publication No. WO/2021/022163, or a pharmaceutically acceptable salt thereof. The compounds thereof are incorporated by reference herein.

In some embodiments of any of the aspects described herein, the compound is a compound described in International Publication No. WO/2020/160198, or a pharmaceutically acceptable salt thereof. The compounds thereof are incorporated by reference herein.

In some embodiments of any of the aspects described herein, the compound is a compound described in International Publication No. WO/2020/160196, or a pharmaceutically acceptable salt thereof. The compounds thereof are incorporated by reference herein.

In some embodiments of any of the aspects described herein, the compound is a compound described in International Publication No. WO/2020/160193, or a pharmaceutically acceptable salt thereof. The compounds thereof are incorporated by reference herein.

In some embodiments of any of the aspects described herein, the compound is a compound described in International Publication No. WO/2020/160192, or a pharmaceutically acceptable salt thereof. The compounds thereof are incorporated by reference herein.

In some embodiments of any of the aspects described herein, the compound is a compound described in International Publication No. WO/2019/152440, or a pharmaceutically acceptable salt thereof. The compounds thereof are incorporated by reference herein.

In some embodiments of any of the aspects described herein, the compound is a compound described in International Publication No. WO/2021/178920, or a pharmaceutically acceptable salt thereof. For example, the compound may be 3-((4-(4-(1-(2,6-dimethoxy-4-(1,4,5-trimethyl-6-oxo-1,6-dihydropyridin-3-yl)benzyl)-3,3-difluoropiperidin-4-yl)piperazin-1-yl)-3-fluorophenyl)amino)piperidine-2,6-dione or a pharmaceutically acceptable salt thereof. For example, the compound may be:

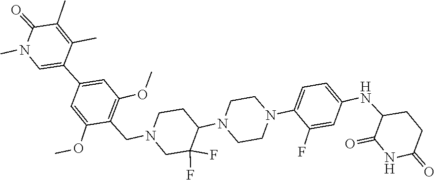 [SMILES: COc1cc(-c2cn(C)c(=O)c(C)c2C)cc(OC)c1CN1CCC(N2CCN(c3ccc(NC4CCC(=O)NC4=O)cc3F)CC2)C(F)(F)C1]

or a pharmaceutically acceptable salt thereof.

In some embodiments of any of the aspects described herein, the compound is a compound described in International Publication No. WO/2021/055295, or a pharmaceutically acceptable salt thereof. For example, the compound is of the following structure:

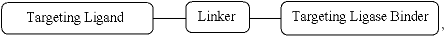

where
the Targeting Ligand is a group that is capable of binding to a bromodomain-containing protein, e.g., BRD9;
the Linker is a group that covalently links the Targeting Ligand to the Targeting Ligase Binder;
the Targeting Ligase Binder is a group that is capable of binding to a ligase (e.g., Cereblon E3 Ubiquitin ligase).

The Targeting Ligand may be, e.g., a group Formula TL-I or TL-II:

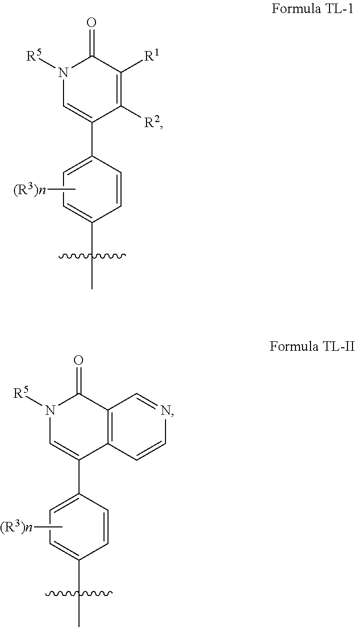

where
n is 0, 1, or 2;
R¹ and R² are independently selected from the group consisting of hydrogen and C₁₋₆ alkyl; or R¹ and R² together with the atoms to which they are attached form an aryl or heteroaryl;
each R³ may be independently selected from the group consisting of C₁₋₆ alkyl, C₁₋₆ alkoxy, and halogen; and
R⁵ is selected from the group consisting of hydrogen and C₁₋₆ alkyl.

The Linker may be, e.g., a group of Formula L-I:

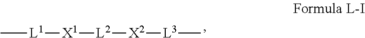

where
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, where * denotes the point of attachment of L¹ to the Targeting Ligand;
X¹ and X² are each independently selected from the group consisting of a bond, carbocyclyl, and heterocyclyl, where the carbocyclyl and heterocyclyl are substituted with 0-4 occurrences of R^{a}, where each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or X¹-L²-X² form a spiroheterocyclyl; and
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O, where * denotes the point of attachment of L³ to X²; where no more than 2 of L¹, X¹, X², L², and L³ can simultaneously be a bond.

The Targeting Ligase Binder may be, e.g., a compound of Formula TLB-I:

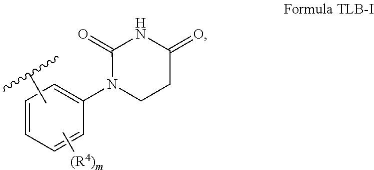

where
R⁴ is selected from the group consisting of C₁₋₆ alkyl, C₁₋₆ alkoxyl, and halogen; and m is 0, 1.

The Targeting Ligase Binder may be, e.g., a compound of Formula TLB-I′:

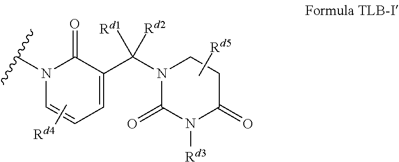

where
R^{d1} and R^{d2} are each independently selected from the group consisting of H, C₁₋₆ alkyl, C₁₋₆ alkoxyl, C₁₋₆ haloalkyl, and C₁₋₆ heteroalkyl;
R^{d3} is H; R^{d4} is selected from the group consisting of H, C1-6 alkyl, halo, C1-6 haloalkyl, and C1-6 heteroalkyl; and
R^{d5} is selected from the group consisting of H, C1-6 alkyl, halo, C₁₋₆ haloalkyl, and C₁₋₆ heteroalkyl.

The BRD9 inhibitor may be, e.g., a compound of the following structure:

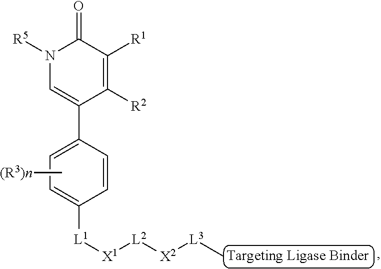

or a pharmaceutically acceptable salt thereof,

where
L¹ is selected from the group consisting of a bond, O, NR′, C(O), C₁₋₆ alkylene, C₁₋₆ heteroalkylene, *C(O)—C₁₋₆ alkylene, C(O)—C₁₋₆ alkenylene*, C₁₋₆ alkenylene, and *C(O)—C₁₋₆ heteroalkylene, where * denotes the point of attachment of L¹ to the Targeting Ligand;
X¹ and X² are each independently selected from the group consisting of a bond, carbocyclyl, and heterocyclyl, where the carbocyclyl and heterocyclyl are substituted with 0-4 occurrences of R^{a}, where each R^{a} is independently selected from the group consisting of C₁₋₆ alkyl, C₁₋₆ alkoxyl, and halogen;
L² is selected from the group consisting of a bond, O, NR′, C₁₋₆ alkylene, and C₁₋₆ heteroalkylene; or X¹-L²-X² form a spiroheterocyclyl; and
L³ is selected from the group consisting of a bond, O, C(O), C₁₋₆ alkylene, C₁₋₆ heteroalkylene, *C(O)—C₁₋₆ alkylene, *C(O)—C₁₋₆ heteroalkylene, and *C(O)—C₁₋₆ alkylene-O, where * denotes the point of attachment of L³ to X².

In some embodiments, no more than 2 of L¹, X¹, X², L², and L³ can simultaneously be a bond.

The compounds found in International Publication Nos. WO/2021/022163, WO/2020/160198, WO/2020/160196, WO/2020/160193, WO/2020/160192, WO/2019/152440, WO/2021/178920, and WO/2021/055295 are incorporated by reference herein.

### Tumor Density

Tumor response has been considered to be pivotal for assessment of therapy efficacy. However, relying on solely the size of a tumor during evaluation for response to cancer therapy has some limitations. Response Evaluation Criteria in Solid Tumors (RECIST) has been the standard method for evaluation of solid tumors for over 20 years and is a set of published guidelines that define when tumors in cancer patients improve, stabilize or progress. RECIST criteria depends on the change in size of target lesions, determined by non-invasive imaging assessment such as a computerized tomography (CT) scan. A revised guideline called modified RECIST criteria (mRECIST) takes into consideration changes in the degree of tumor arterial enhancement. Table 1 below describes RECIST criteria.

TABLE 1
RECIST Criteria for Tumor Response to Therapy

| Category | Criteria |
|---|---|
| Complete Response (CR) | disappearance of all target lesions, with any |
| | pathological lymph nodes (target or non-target) |
| | having a reduction in short axis to <10 mm |
| Partial Response (PR) | at least a 30% decrease in the sum of diameters |
| | of target lesions, taking as reference the baseline |
| | sum diameters |
| Stable Disease (SD) | neither sufficient shrinkage to qualify for partial |
| | response, nor sufficient increase to qualify for |
| | progressive disease, taking as reference the |
| | smallest sum diameters while on study. |
| Progressive Disease (PD) | at least a 20% increase in the sum of diameters |
| | of target lesions, taking as reference the |
| | smallest sum on study. |
| | In addition, the sum must also demonstrate an |
| | absolute increase of at least 5 mm. |
| | appearance of one or more new lesions |

Some cancer treatments may lead to disease stabilization without actual shrinkage of tumor size, in which progress is missed using RECIST and/or mRECIST criteria. Therefore, evaluation based on tumor size alone may no longer be adequate for modern tumor therapy follow-up. Choi et al developed new criteria typically referred to as “Choi criteria”. This set of criteria was originally developed for gastrointestinal stromal tumors (GIST). Choi criteria assesses a change in size or a change in density of target lesions, which varies from RECIST and mRECIST criteria. Choi criteria appear to be better predictors of clinical response in some tumors such as GIST. “Choi” criteria refers to a set of computed tomography response criteria originally described by Choi et al. (J. Clin. Oncol. 2007, 25(13):1753-1759) and evaluates a change in the size or in the density of target lesions as measured by CT. The Choi criteria also categorize patients using the CR, PR, SD and PD groupings (Table 2).

TABLE 2
Choi Criteria for Tumor Response to Therapy

| Category | Criteria |
|---|---|
| Complete Response (CR) | a disappearance of all target lesions |
| | no new lesions |
| Partial Response (PR) | decrease in tumor size of greater than or equal |
| | to 10% and/or a decrease in tumor density |
| | (HU) of greater than or equal to 15% according |
| | to a CT scan |
| | no new lesions |
| | no obvious progression of non-measurable |
| | disease or non-target lesions |
| Stable Disease (SD) | neither sufficient shrinkage to qualify for PR, |
| | nor sufficient increase to qualify for PD, |
| | taking as reference the smallest sum diameters |
| | while on study as defined in RECIST. |
| | no symptomatic deterioration attributed to |
| | tumor progression |
| Progressive Disease (PD) | An increase in tumor size of greater than or |
| | equal to 10% and does not meet criteria of PR |
| | by tumor density (HU) on CT |
| | New lesions |
| | New intratumoral nodules or increase in the |
| | size of existing intratumoral nodules |

In Choi criteria, tumor size is represented by the sum of the longest cross-sectional diameters for target lesions, similarly to RECIST criteria. First proposed for metastatic renal cell cancer treated with tyrosine kinase inhibitors, a modified Choi criteria called mChoi has one major difference from standard Choi criteria in that both a greater than or equal to 10% decrease in tumor size and a greater than or equal to 15% tumor attenuation according to CT scan are required for the diagnosis of partial response. In this setting the mChoi system was found to correlate better with time to disease progression.

In Choi criteria, tumor density is determined by measuring CT tumor attenuation coefficient (Hounsfield unit [HU]). The CT tumor attenuation coefficient (density) of each tumor is measured in HU by drawing a region of interest around the margin of the entire tumor. In cases in which subjects are scanned with triphasic techniques, the portal venous phase can be used for the tumor density measurement. Tumor density measurements of all lesions are combined and a mean HU for each patient can be computed.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to decrease a BRD9 immunohistochemistry score of the subject. In some embodiments of any of the aspects disclosed herein, the effective amount is an amount sufficient to cause a greater than or equal to 10% decrease in tumor size and/or a greater than or equal to 15% decrease in tumor attenuation in the subject. In some embodiments of any of the aspects disclosed herein, the subject has no new lesions. In some embodiments of any of the aspects disclosed herein, the subject has no obvious progression of non-measurable disease.

In one aspect, the present disclosure provides a method of decreasing a BRD9 immunohistochemistry score in a subject, the method comprising administering an effective amount of a compound that reduces the level and/or activity of BRD9, or a pharmaceutically acceptable salt thereof. In some embodiments of any of the aspects disclosed herein, the effective amount is an amount sufficient to cause a greater than or equal to 10% decrease in tumor size and/or a greater than or equal to 15% decrease in tumor attenuation in the subject. In some embodiments of any of the aspects disclosed herein, the subject has no new lesions. In some embodiments of any of the aspects disclosed herein, the subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to reduce the level of BRD9 expression in the subject. In some embodiments of any of the aspects disclosed herein, the effective amount is an amount sufficient to cause a greater than or equal to 10% decrease in tumor size and/or a greater than or equal to 15% decrease in tumor attenuation in the subject. In some embodiments of any of the aspects disclosed herein, the subject has no new lesions. In some embodiments of any of the aspects disclosed herein, the subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation. In some embodiments of any of the aspects disclosed herein, the subject has no new lesions. In some embodiments of any of the aspects disclosed herein, subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size or a greater than or equal to 15% decrease in tumor attenuation. In some embodiments of any of the aspects disclosed herein, the subject has no new lesions. In some embodiments of any of the aspects disclosed herein, subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and a greater than or equal to 15% decrease in tumor attenuation. In some embodiments of any of the aspects disclosed herein, the subject has no new lesions. In some embodiments of any of the aspects disclosed herein, subject has no obvious progression of non-measurable disease.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation, no progression of non-target lesions, and no new lesions.

In some embodiments of any of the aspects disclosed herein, the subject is evaluated for disease progression using Choi criteria.

In some embodiments of any of the aspects disclosed herein, the subject is evaluated for disease progression using mChoi criteria.

In some embodiments of any of the aspects disclosed herein, the subject has a partial response to cancer therapy.

In some embodiments of any of the aspects disclosed herein, tumor size and/or tumor attenuation of the subject is measured by computed tomography (CT).

In some embodiments of any of the aspects disclosed herein, the subject has a disappearance of all target lesions.

In some embodiments of any of the aspects disclosed herein, the subject has no new lesions.

In some embodiments of any of the aspects disclosed herein, the subject has a complete response to cancer therapy.

In some embodiments of any of the aspects disclosed herein, the subject has stabilized disease in response to cancer therapy.

### Pharmaceutical Uses

The compounds described herein are useful in the methods of the disclosure and, while not bound by theory, are believed to exert their desirable effects through their ability to modulate the level, status, and/or activity of a BAF complex, e.g., by inhibiting the activity or level of the BRD9 protein in a cell within the BAF complex in a mammal.

An aspect of the present disclosure relates to methods of treating disorders related to BRD9 such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present disclosure in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

In some embodiments of any of the aspects described herein, the cancer is a malignant rhabdoid tumor, a CDS+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma, non-small cell lung cancer, stomach cancer, breast cancer, or SMARCAB1-loss cancer.

In some embodiments, the cancer is a malignant rhabdoid tumor.

In some embodiments, the cancer is a CDS+ T-cell lymphoma.

In some embodiments, the cancer is endometrial carcinoma.

In some embodiments, the cancer is ovarian carcinoma.

In some embodiments, the cancer is bladder cancer.

In some embodiments, the cancer is stomach cancer.

In some embodiments, the cancer is pancreatic cancer.

In some embodiments, the cancer is esophageal cancer.

In some embodiments, the cancer is prostate cancer.

In some embodiments, the cancer is renal cell carcinoma.

In some embodiments, the cancer is melanoma.

In some embodiments, the cancer is colorectal cancer.

In some embodiments, the cancer is a sarcoma, e.g., a soft tissue sarcoma, synovial sarcoma, Ewing's sarcoma, osteosarcoma, rhabdomyosarcoma, adult fibrosarcoma, alveolar soft-part sarcoma, angiosarcoma, clear cell sarcoma, desmoplastic small round cell tumor, epithelioid sarcoma, fibromyxoid sarcoma, gastrointestinal stromal tumor, Kaposi sarcoma, liposarcoma, leiomyosarcoma, malignant mesenchymoma malignant peripheral nerve sheath tumors, myxofibrosarcoma, or low-grade rhabdomyosarcoma. In particular embodiments, the sarcoma is synovial sarcoma.

In some embodiments, the cancer is non-small cell lung cancer.

In some embodiments, the cancer is breast cancer.

In some embodiments, the cancer is a SMARCAB1-loss cancer, e.g., high grade ovarian serous adenocarcinoma, epithelioid sarcoma, atypical teratoid/rhabdoid tumor, prostate adenocarcinoma, conventional glioblastoma multiforme, breast invasive ductal carcinoma, sarcoma (NOS), gastrointestinal stromal tumor, kidney medullary carcinoma, pancreatic neuroendocrine neoplasm, sinonasal adenocarcinoma, adrenal cortex carcinoma, proximal-type epithelioid sarcoma, head and neck carcinoma, invasive breast carcinoma, rhabdoid tumor of the kidney, dedifferentiated liposarcoma, pancreatic adenocarcinoma, chordoma, testicular yolk sac tumor, small cell lung carcinoma, squamous cell lung carcinoma, acral lentiginous melanoma, peripheral T-cell lymphoma, nasal cavity and paranasal sinus squamous cell carcinoma, malignant peripheral nerve sheath tumor, breast invasive lobular carcinoma, chordoma, a SMARCB1-mutant cancer, or lung adenocarcinoma. In particular embodiments, the SMARCAB1-loss cancer is chordoma.

### Dosages

The dosage (e.g., effective amount) of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response.

In the context of treating cancer or decreasing a BRD9 immunohistochemistry score in a subject, the effective amount of a compound of the present disclosure or a pharmaceutically acceptable salt thereof may be an amount sufficient to decrease a BRD9 immunohistochemistry score of the subject. BRD9 immunohistochemistry score may be determined using techniques known in the art, e.g., according to the principles described in Ishibashi et al., J. Clin. Endocrinol. Metab., 88:2309-2317, 2003, the disclosure of which is incorporated herein.

In some embodiments, the decrease in BRD9 immunohistochemistry score in the subject is compared to a BRD9 immunohistochemistry score in the subject prior to administration of the compound of the present disclosure or pharmaceutically acceptable salt thereof. In some embodiments, the decrease in BRD9 immunohistochemistry score in the subject is compared to a reference BRD9 immunohistochemistry score. In some embodiments, the decrease in BRD9 immunohistochemistry score in the subject is compared to a BRD9 immunohistochemistry score in a subject who was not administered the compound of the present disclosure or pharmaceutically acceptable salt thereof.

In some embodiments, the BRD9 immunohistochemistry score is an H score.

In some embodiments, the effective amount of a compound of the present disclosure or a pharmaceutically acceptable salt thereof is an amount sufficient to reduce a BRD9 immunohistochemistry score to a score of between about 15 and about 0.

The BRD9 immunohistochemistry score may be determined for a tumor in the subject. Accordingly, in some embodiments, the effective amount of a compound of the present disclosure or a pharmaceutically acceptable salt thereof is an amount sufficient to decrease a BRD9 immunohistochemistry score of a tumor in the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%), compared to the BRD9 immunohistochemistry score in the tumor prior to administration of the compound of the present disclosure or pharmaceutically acceptable salt thereof. Accordingly, in some embodiments, the effective amount of a compound of the present disclosure or a pharmaceutically acceptable salt thereof is an amount sufficient to decrease a BRD9 immunohistochemistry score of a tumor in the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%), compared to the BRD9 immunohistochemistry score in the tumor prior to administration of the compound of the present disclosure or pharmaceutically acceptable salt thereof.

In some embodiments, the effective amount of a compound of the present disclosure or a pharmaceutically acceptable salt thereof is an amount sufficient to maintain a BRD9 immunohistochemistry score that is about 10 and about 0 in the subject over at least 14 days (e.g., at least 21 days or at least 28 days).

In the context of treating cancer or maintaining a reduction in levels of BRD9 expression in a subject, the effective amount of a compound of the present disclosure or a pharmaceutically acceptable salt thereof may be an amount sufficient to reduce the level of BRD9 expression in the subject.

In some embodiments, the reduction in the level of BRD9 expression in the subject is compared to the level of BRD expression in the subject prior to administration of the compound of the present disclosure or pharmaceutically acceptable salt thereof. In some embodiments, the reduction in the level of BRD9 expression in the subject is a compared to the level of BRD9 expression in a reference. In some embodiments, the reduction in the level of BRD9 expression in the subject is compared to the level of BRD9 expression in a subject who was not administered the compound of the present disclosure or pharmaceutically acceptable salt thereof.

The level of BRD expression may be determined in a blood sample of the subject. Accordingly, in some embodiments, the effective amount of a compound of the present disclosure or a pharmaceutically acceptable salt thereof is an amount sufficient to reduce the level of BRD9 expression in a blood sample of the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the level of BRD9 expression in a blood sample of the subject prior to administration of the compound of the present disclosure or pharmaceutically acceptable salt thereof. Accordingly, in some embodiments, the effective amount of a compound of the present disclosure or a pharmaceutically acceptable salt thereof is an amount sufficient to reduce the level of BRD9 expression in a blood sample of the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the level of BRD9 expression in a blood sample of the subject prior to administration of the compound of the present disclosure or pharmaceutically acceptable salt thereof.

The level of BRD expression may also be determined in a tumor of the subject. Accordingly, in some embodiments, the effective amount of a compound of the present disclosure or a pharmaceutically acceptable salt thereof is an amount sufficient to reduce the level of BRD9 expression in a tumor of the subject by between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) compared to the level of BRD9 expression in the tumor the subject prior to administration of the compound of the present disclosure or pharmaceutically acceptable salt thereof. Accordingly, in some embodiments, the effective amount of a compound of the present disclosure or a pharmaceutically acceptable salt thereof is an amount sufficient to reduce the level of BRD9 expression in a tumor of the subject by at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) compared to the level of BRD9 expression in the tumor the subject prior to administration of the compound of the present disclosure or pharmaceutically acceptable salt thereof.

In some embodiments, the effective amount of a compound of the present disclosure or a pharmaceutically acceptable salt thereof is an amount sufficient to maintain a reduction of BRD9 expression of between about 90% and about 100% (e.g., between about 91% and about 100%, between about 92% and about 100%, between about 93% and about 100%, between about 94% and about 100%, between about 95% and about 100%, between about 96% and about 100%, between about 97% and about 100%, between about 98% and about 100%, or between about 99% and about 100%) in the subject over at least 14 days (e.g., at least 21 days or at least 28 days). In some embodiments, the effective amount of a compound of the present disclosure or a pharmaceutically acceptable salt thereof is an amount sufficient to maintain a reduction of BRD9 expression of at least about 90% (e.g., at least about 91%, at least about 92%, at least about 93%, at least about 94%, at least about 95%, at least about 96%, at least about 97%, at least about 98%, at least about 99%; e.g., by about 90%, about 91%, about 92%, about 93%, about 94%, about 95%, about 96%, about 97%, about 98%, or about 99%) in the subject over at least 14 days (e.g., at least 21 days or at least 28 days).

In some embodiments, the effective amount of a compound of the present disclosure or a pharmaceutically acceptable salt thereof is administered in a dose of 20-80 mg/kg (e.g., 20-60 mg/kg, 20-40 mg/kg, 40-80 mg/kg, 40-60 mg/kg, or 60-80 mg/kg).

In some embodiments, the effective amount of a compound of the present disclosure or a pharmaceutically acceptable salt thereof is administered in a dose of 20 mg/kg. In some embodiments, the effective amount of a compound of the present disclosure or a pharmaceutically acceptable salt thereof is administered in a dose of 40 mg/kg. In some embodiments, the effective amount of a compound of the present disclosure or a pharmaceutically acceptable salt thereof is administered in a dose of 60 mg/kg. In some embodiments, the effective amount of a compound of the present disclosure or a pharmaceutically acceptable salt thereof is administered in a dose of 80 mg/kg.

In some embodiments, the effective amount of a compound of the present disclosure or a pharmaceutically acceptable salt thereof is administered at least once per week.

In some embodiments, the effective amount of a compound of the present disclosure or a pharmaceutically acceptable salt thereof is administered at least twice per week.

In some embodiments, the effective amount of a compound of the present disclosure or a pharmaceutically acceptable salt thereof is administered in a dose of 20 mg/kg once per week. In some embodiments, the effective amount of a compound of the present disclosure or a pharmaceutically acceptable salt thereof is administered in a dose of 40 mg/kg once per week. In some embodiments, the effective amount of a compound of the present disclosure or a pharmaceutically acceptable salt thereof is administered in a dose of 60 mg/kg once per week. In some embodiments, the effective amount of a compound of the present disclosure or a pharmaceutically acceptable salt thereof is administered in a dose of 80 mg/kg once per week.

In some embodiments, the effective amount of a compound of the present disclosure or a pharmaceutically acceptable salt thereof is administered in a dose of 20 mg/kg twice per week. In some embodiments, the effective amount of a compound of the present disclosure or a pharmaceutically acceptable salt thereof is administered in a dose of 40 mg/kg twice per week. In some embodiments, the effective amount of a compound of the present disclosure or a pharmaceutically acceptable salt thereof is administered in a dose of 60 mg/kg twice per week. In some embodiments, the effective amount of a compound of the present disclosure or a pharmaceutically acceptable salt thereof is administered in a dose of 80 mg/kg twice per week.

In some embodiments, the effective amount of a compound of the present disclosure of a pharmaceutically acceptable salt thereof is administered to the subject in a 14-day dosing cycle.

In some embodiments, the effective amount of a compound of the present disclosure of a pharmaceutically acceptable salt thereof is administered to the subject in a 2 week dosing cycle.

In some embodiments, the effective amount of a compound of the present disclosure of a pharmaceutically acceptable salt thereof is administered to the subject in a 21-day dosing cycle.

In some embodiments, the effective amount of a compound of the present disclosure of a pharmaceutically acceptable salt thereof is administered to the subject in a 3 week dosing cycle.

In some embodiments, the effective amount of a compound of the present disclosure of a pharmaceutically acceptable salt thereof is administered to the subject in a 28-day dosing cycle.

In some embodiments, the effective amount of a compound of the present disclosure of a pharmaceutically acceptable salt thereof is administered to the subject in a 4 week dosing cycle.

In some embodiments, the effective amount of a compound of the present disclosure of a pharmaceutically acceptable salt thereof is administered to the subject in a one month dosing cycle.

In some embodiments, a formulation of the present disclosure is administered in an amount sufficient to maintain a concentration of at least 1 ng/mL of compound for at least 14 days after administration. In some embodiments, a formulation of the present disclosure is administered in an amount sufficient to maintain a concentration of at least 1 ng/mL of compound for at least 2 weeks after administration. In some embodiments, a formulation of the present disclosure is administered in an amount sufficient to maintain a concentration of at least 1 ng/mL of compound for at least 21 days after administration. In some embodiments, a formulation of the present disclosure is administered in an amount sufficient to maintain a concentration of at least 1 ng/mL of compound for at least 3 weeks after administration. In some embodiments, a formulation of the present disclosure is administered in an amount sufficient to maintain a concentration of at least 1 ng/mL of compound for at least 28 days after administration. In some embodiments, a formulation of the present disclosure is administered in an amount sufficient to maintain a concentration of at least 1 ng/mL of compound for at least 4 weeks after administration. In some embodiments, a formulation of the present disclosure is administered in an amount sufficient to maintain a concentration of at least 1 ng/mL of compound for at least 1 month after administration.

In some embodiments, a formulation of the present disclosure is administered in an amount sufficient to maintain a concentration of at least 2 ng/mL of compound for at least 14 days after administration. In some embodiments, a formulation of the present disclosure is administered in an amount sufficient to maintain a concentration of at least 2 ng/mL of compound for at least 2 weeks after administration. In some embodiments, a formulation of the present disclosure is administered in an amount sufficient to maintain a concentration of at least 2 ng/mL of compound for at least 21 days after administration. In some embodiments, a formulation of the present disclosure is administered in an amount sufficient to maintain a concentration of at least 2 ng/mL of compound for at least 3 weeks after administration. In some embodiments, a formulation of the present disclosure is administered in an amount sufficient to maintain a concentration of at least 2 ng/mL of compound for at least 28 days after administration. In some embodiments, a formulation of the present disclosure is administered in an amount sufficient to maintain a concentration of at least 2 ng/mL of compound for at least 4 weeks after administration. In some embodiments, a formulation of the present disclosure is administered in an amount sufficient to maintain a concentration of at least 2 ng/mL of compound for at least 1 month after administration.

In some embodiments, a formulation of the present disclosure is administered in an amount sufficient to maintain a concentration of at least 3 ng/mL of compound for at least 14 days after administration. In some embodiments, a formulation of the present disclosure is administered in an amount sufficient to maintain a concentration of at least 3 ng/mL of compound for at least 2 weeks after administration. In some embodiments, a formulation of the present disclosure is administered in an amount sufficient to maintain a concentration of at least 3 ng/mL of compound for at least 21 days after administration. In some embodiments, a formulation of the present disclosure is administered in an amount sufficient to maintain a concentration of at least 3 ng/mL of compound for at least 3 weeks after administration. In some embodiments, a formulation of the present disclosure is administered in an amount sufficient to maintain a concentration of at least 3 ng/mL of compound for at least 28 days after administration. In some embodiments, a formulation of the present disclosure is administered in an amount sufficient to maintain a concentration of at least 3 ng/mL of compound for at least 4 weeks after administration. In some embodiments, a formulation of the present disclosure is administered in an amount sufficient to maintain a concentration of at least 3 ng/mL of compound for at least 1 month after administration.

In some embodiments, a formulation of the present disclosure is administered in an amount sufficient to maintain a concentration of at least 5 ng/mL of compound for at least 14 days after administration. In some embodiments, a formulation of the present disclosure is administered in an amount sufficient to maintain a concentration of at least 5 ng/mL of compound for at least 2 weeks after administration. In some embodiments, a formulation of the present disclosure is administered in an amount sufficient to maintain a concentration of at least 5 ng/mL of compound for at least 21 days after administration. In some embodiments, a formulation of the present disclosure is administered in an amount sufficient to maintain a concentration of at least 5 ng/mL of compound for at least 3 weeks after administration. In some embodiments, a formulation of the present disclosure is administered in an amount sufficient to maintain a concentration of at least 5 ng/mL of compound for at least 28 days after administration. In some embodiments, a formulation of the present disclosure is administered in an amount sufficient to maintain a concentration of at least 5 ng/mL of compound for at least 4 weeks after administration. In some embodiments, a formulation of the present disclosure is administered in an amount sufficient to maintain a concentration of at least 5 ng/mL of compound for at least 1 month after administration.

In some embodiments, a formulation of the present disclosure is administered in an amount sufficient to maintain a concentration of at least 10 ng/mL of compound for at least 14 days after administration. In some embodiments, a formulation of the present disclosure is administered in an amount sufficient to maintain a concentration of at least 10 ng/mL of compound for at least 2 weeks after administration. In some embodiments, a formulation of the present disclosure is administered in an amount sufficient to maintain a concentration of at least 10 ng/mL of compound for at least 21 days after administration. In some embodiments, a formulation of the present disclosure is administered in an amount sufficient to maintain a concentration of at least 10 ng/mL of compound for at least 3 weeks after administration. In some embodiments, a formulation of the present disclosure is administered in an amount sufficient to maintain a concentration of at least 10 ng/mL of compound for at least 28 days after administration. In some embodiments, a formulation of the present disclosure is administered in an amount sufficient to maintain a concentration of at least 10 ng/mL of compound for at least 4 weeks after administration. In some embodiments, a formulation of the present disclosure is administered in an amount sufficient to maintain a concentration of at least 10 ng/mL of compound for at least 1 month after administration.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to decrease a BRD9 immunohistochemistry score of the subject. In some embodiments of any of the aspects disclosed herein, the effective amount is an amount sufficient to maintain a plasma concentration of 3 ng/mL of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days.

In one aspect, the present disclosure provides a method of decreasing a BRD9 immunohistochemistry score in a subject, the method comprising administering an effective amount of a compound that reduces the level and/or activity of BRD9, or a pharmaceutically acceptable salt thereof. In some embodiments of any of the aspects disclosed herein, the effective amount is an amount sufficient to maintain a plasma concentration of 3 ng/mL of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to reduce the level of BRD9 expression in the subject. In some embodiments of any of the aspects disclosed herein, the effective amount is an amount sufficient to maintain a plasma concentration of 3 ng/mL of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to cause a greater than or equal to 10% decrease in tumor size and/or a greater than or equal to 15% decrease in tumor attenuation. In some embodiments of any of the aspects disclosed herein, the effective amount is an amount sufficient to maintain a plasma concentration of 3 ng/mL of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days.

In one aspect, the present disclosure provides a method of treating cancer in a subject in need thereof, the method comprising administering an effective amount of a compound that reduces the level and/or activity of BRD9, or a pharmaceutically acceptable salt thereof, wherein the effective amount is an amount sufficient to maintain a plasma concentration of 3 ng/mL of the compound in a subject. In some embodiments of any of the aspects disclosed herein, the effective amount is an amount sufficient to maintain a plasma concentration of 3 ng/mL of the compound in a subject, over at least 14 days. In some embodiments of any of the aspects disclosed herein, the effective amount is an amount sufficient to maintain a plasma concentration of 3 ng/mL of the compound in a subject, over at least 21 days. In some embodiments of any of the aspects disclosed herein, the effective amount is an amount sufficient to maintain a plasma concentration of 3 ng/mL of the compound in a subject, over at least 28 days.

### Combination Therapies

A method of the disclosure may include administering to the subject in need thereof an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Also included is 5-fluorouracil (5-FU), leucovorin (LV), irinotecan, oxaliplatin, capecitabine, paclitaxel, and docetaxel. Non-limiting examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomycin, actinomycin, authramycin, azaserine, bleomycin, cactinomycin, carubicin, carzinophilin, chromomycin, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocin; mitoguazone; mitoxantrone; mopidamol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verrucarin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® docetaxel (Rhone-Poulenc Rorer, Antony, France); chlorambucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a DNA damaging agent (e.g., a platinum-based antineoplastic agent, topoisomerase inhibitors, PARP inhibitors, alkylating antineoplastic agents, and ionizing radiation).

Examples of platinum-based antineoplastic agent that may be used as a second therapeutic agent in the compositions and methods of the disclosure are cisplatin, carboplatin, oxaliplatin, dicycloplatin, heptaplatin, lobaplatin, miriplatin, nedaplatin, triplatin tetranitrate, phenanthriplatin, picoplatin, and satraplatin. In some embodiments, the second therapeutic agent is cisplatin and the treated cancer is a testicular cancer, ovarian cancer, or a bladder cancer (e.g., advanced bladder cancer). In some embodiments, the second therapeutic agent is carboplatin and the treated cancer is an ovarian cancer, lung cancer, head and neck cancer, brain cancer, or neuroblastoma. In some embodiments, the second therapeutic agent is oxaliplatin and the treated cancer is a colorectal cancer. In some embodiments, the second therapeutic agent is dicycloplatin and the treated cancer is a non-small cell lung cancer or prostate cancer. In some embodiments, the second therapeutic agent is heptaplatin and the treated cancer is a gastric cancer. In some embodiments, the second therapeutic agent is lobaplatin and the treated cancer is a breast cancer. In some embodiments, the second therapeutic agent is miriplatin and the treated cancer is a hepatocellular carcinoma. In some embodiments, the second therapeutic agent is nedaplatin and the treated cancer is a nasopharyngeal carcinoma, esophageal cancer, squamous cell carcinoma, or cervical cancer. In some embodiments, the second therapeutic agent is triplatin tetranitrate and the treated cancer is a lung cancer (e.g., small cell lung cancer) or pancreatic cancer. In some embodiments, the second therapeutic agent is picoplatin and the treated cancer is a lung cancer (e.g., small cell lung cancer), prostate cancer, bladder cancer, or colorectal cancer. In some embodiments, the second therapeutic agent is satraplatin and the treated cancer is a prostate cancer, breast cancer, or lung cancer.

Examples of topoisomerase inhibitors that may be used as a second therapeutic agent in the compositions and methods of the disclosure are etoposide, teniposide, doxorubicin, daunorubicin, mitoxantrone, amsacrine, ellipticine, irinotecan, topotecan, camptothecin, and diflomotecan. In some embodiments, the second therapeutic agent is etoposide and the treated cancer is a lung cancer (e.g., small cell lung cancer) or testicular cancer. In some embodiments, the second therapeutic agent is teniposide and the treated cancer is an acute lymphoblastic leukemia (e.g., childhood acute lymphoblastic leukemia). In some embodiments, the second therapeutic agent is doxorubicin and the treated cancer is an acute lymphoblastic leukemia, acute myeloblastic leukemia, Hodgkin's lymphoma, non-Hodgkin's lymphoma, breast cancer, Wilms tumor, neuroblastoma, soft tissue sarcoma, bone sarcomas, ovarian carcinoma, transitional cell bladder carcinoma, thyroid carcinoma, gastric carcinoma, or bronchogenic carcinoma. In some embodiments, the second therapeutic agent is daunorubicin and the treated cancer is an acute lymphoblastic leukemia or acute myeloid leukemia. In some embodiments, the second therapeutic agent is mitoxantrone and the treated cancer is a prostate cancer or acute nonlymphocytic leukemia. In some embodiments, the second therapeutic agent is amsacrine and the treated cancer is a leukemia (e.g., acute adult leukemia). In some embodiments, the second therapeutic agent is irinotecan and the treated cancer is a colorectal cancer. In some embodiments, the second therapeutic agent is topotecan and the treated cancer is a lung cancer (e.g., small cell lung cancer). In some embodiments, the second therapeutic agent is diflomotecan and the treated cancer is a lung cancer (e.g., small cell lung cancer).

Examples of alkylating antineoplastic agents that may be used as a second therapeutic agent in the compositions and methods of the disclosure are cyclophosphamide, uramustine, melphalan, chlorambucil, ifosfamide, bendamustine, carmustine, lomustine, chlorozotocin, fotemustine, nimustine, ranimustine, busulfan, improsulfan, piposulfan, chlornaphazine, cyclophosphamide, estramustine, mechlorethamine, mechlorethamine oxide hydrochloride, novembichin, phenesterine, prednimustine, trofosfamide, procarbazine, altretamine, dacarbazine, mitozolomide, and temozolomide. In some embodiments, the second therapeutic agent is cyclophosphamide and the treated cancer is a non-Hodgkin's lymphoma. In some embodiments, the second therapeutic agent is melphalan and the treated cancer is a multiple myeloma, ovarian cancer, or melanoma. In some embodiments, the second therapeutic agent is chlorambucil and the treated cancer is a chronic lymphatic leukemia, malignant lymphoma (e.g., lymphosarcoma, giant follicular lymphoma, or Hodgkin's lymphoma). In some embodiments, the second therapeutic agent is ifosfamide and the treated cancer is a testicular cancer. In some embodiments, the second therapeutic agent is bendamustine and the treated cancer is a chronic lymphocytic leukemia or non-Hodgkin's lymphoma. In some embodiments, the second therapeutic agent is carmustine and the treated cancer is a brain cancer (e.g., glioblastoma, brainstem glioma, medulloblastoma, astrocytoma, ependymoma, or a metastatic brain tumor), multiple myeloma, Hodgkin's disease, or non-Hodgkin's lymphoma. In some embodiments, the second therapeutic agent is lomustine and the treated cancer is a brain cancer or Hodgkin's lymphoma. In some embodiments, the second therapeutic agent is fotemustine and the treated cancer is a melanoma. In some embodiments, the second therapeutic agent is nimustine and the treated cancer is a brain cancer. In some embodiments, the second therapeutic agent is ranimustine and the treated cancer is a chronic myelogenous leukemia or polycythemia vera. In some embodiments, the second therapeutic agent is busulfan and the treated cancer is a chronic myelogenous leukemia. In some embodiments, the second therapeutic agent is improsulfan and the treated cancer is a sarcoma. In some embodiments, the second therapeutic agent is estramustine and the treated cancer is a prostate cancer (e.g., prostate carcinoma). In some embodiments, the second therapeutic agent is mechlorethamine and the treated cancer is a cutaneous T-cell lymphoma. In some embodiments, the second therapeutic agent is trofosfamide and the treated cancer is a sarcoma (e.g., soft tissue sarcoma). In some embodiments, the second therapeutic agent is procarbazine and the treated cancer is a Hodgkin's disease. In some embodiments, the second therapeutic agent is altretamine and the treated cancer is an ovarian cancer. In some embodiments, the second therapeutic agent is dacarbazine and the treated cancer is a melanoma, Hodgkin's lymphoma, or sarcoma. In some embodiments, the second therapeutic agent is temozolomide and the treated cancer is a brain cancer (e.g., astrocytoma or glioblastoma) or lung cancer (e.g., small cell lung cancer).

Examples of PARP inhibitors that may be used as a second therapeutic agent in the compositions and methods of the disclosure are niraparib, olaparib, rucaparib, talazoparib, veliparib, pamiparib, CK-102, or E7016. Advantageously, the compounds of the disclosure and a DNA damaging agent may act synergistically to treat cancer. In some embodiments, the second therapeutic agent is niraparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is olaparib and the treated cancer is a lung cancer (e.g., small cell lung cancer), ovarian cancer (e.g., BRCA mutated ovarian cancer), breast cancer (e.g., BRCA mutated breast cancer), fallopian tube cancer (e.g., BRCA mutated fallopian tube cancer), primary peritoneal cancer (e.g., BRCA mutated primary peritoneal cancer), prostate cancer (e.g., castration-resistant prostate cancer), or pancreatic cancer (e.g., pancreatic adenocarcinoma). In some embodiments, the second therapeutic agent is rucaparib and the treated cancer is an ovarian cancer (e.g., BRCA mutated ovarian cancer), fallopian tube cancer (e.g., BRCA mutated fallopian tube cancer), or primary peritoneal cancer (e.g., BRCA mutated primary peritoneal cancer). In some embodiments, the second therapeutic agent is talazoparib and the treated cancer is a breast cancer (e.g., BRCA mutated breast cancer). In some embodiments, the second therapeutic agent is veliparib and the treated cancer is a lung cancer (e.g., non-small cell lung cancer), melanoma, breast cancer, ovarian cancer, prostate cancer, or brain cancer. In some embodiments, the second therapeutic agent is pamiparib and the treated cancer is an ovarian cancer. In some embodiments, the second therapeutic agent is CK-102 and the treated cancer is a lung cancer (e.g., non-small cell lung cancer). In some embodiments, the second therapeutic agent is E7016 and the treated cancer is a melanoma.

Without wishing to be bound by theory, the synergy between the compounds of the disclosure and DNA damaging agents may be attributed to the necessity of BRD9 for DNA repair; inhibition of BRD9 may sensitize cancer (e.g., cancer cell or cancer tissue) to DNA damaging agents.

In some embodiments, the second therapeutic agent is a JAK inhibitor (e.g., JAK1 inhibitor). Non-limiting examples of JAK inhibitors that may be used as a second therapeutic agent in the compositions and methods of the disclosure include tofacitinib, ruxolitinib, oclacitinib, baricitinib, peficitinib, fedratinib, upadacitinib, filgotinib, cerdulatinib, gandotinib, lestaurtinib, momelotinib, pacritinib, abrocitinib, solcitinib, itacitinib, or SHR0302. Without wishing to be bound by theory, the synergy between the compounds of the disclosure and JAK inhibitors may be inhibitor of SAGA complex to their combined effect of downregulating Foxp3+ Treg cells. In some embodiments, the second therapeutic agent is ruxolitinib and the treated cancer is a myeloproliferative neoplasm (e.g., polycythemia or myelofibrosis), ovarian cancer, breast cancer, pancreatic cancer. In some embodiments, the second therapeutic agent is fedratinib and the treated cancer is a myeloproliferative neoplasm (e.g., myelofibrosis). In some embodiments, the second therapeutic agent is cerdulatinib and the treated cancer is a lymphoma (e.g., peripheral T-cell lymphoma). In some embodiments, the second therapeutic agent is gandotinib and the treated cancer is a myeloproliferative neoplasm (e.g., polycythemia or myelofibrosis). In some embodiments, the second therapeutic agent is lestaurtinib and the treated cancer is a myeloproliferative neoplasm (e.g., polycythemia or myelofibrosis), leukemia (e.g., acute myeloid leukemia), pancreatic cancer, prostate cancer, or neuroblastoma. In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma). In some embodiments, the second therapeutic agent is momelotinib and the treated cancer is a myeloproliferative neoplasm (e.g., polycythemia or myelofibrosis). In some embodiments, the second therapeutic agent is momelotinib and the treated cancer is a myeloproliferative neoplasm (e.g., polycythemia or myelofibrosis) or pancreatic cancer (e.g., pancreatic ductal adenocarcinoma).

In some embodiments, the second therapeutic agent is an inhibitor of SAGA complex or a component thereof. A SAGA complex inhibitor may be, e.g., an inhibitory antibody or small molecule inhibitor, of CCDC101, Tada2B, Tada3, Usp22, Tada1, Taf6I, Supt5, Supt20, or a combination thereof. Without wishing to be bound by theory, the synergy between the compounds of the disclosure and inhibitors of SAGA complex may be attributed to their combined effect of downregulating Foxp3+ Treg cells. In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOLIRIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the second therapeutic agent is ipilimumab and the treated cancer is a melanoma, kidney cancer, lung cancer (e.g., non-small cell lung cancer or small cell lung cancer), or prostate cancer. In some embodiments, the second therapeutic agent is tremelimumab and the treated cancer is a melanoma, mesothelioma, or lung cancer (e.g., non-small cell lung cancer). In some embodiments, the second therapeutic agent is nivolumab and the treated cancer is a melanoma, lung cancer (e.g., non-small cell lung cancer or small cell lung cancer), kidney cancer, Hodgkin's lymphoma, head and neck cancer (e.g., squamous cell carcinoma of the head and neck), urothelial carcinoma, hepatocellular carcinoma, or colorectal cancer. In some embodiments, the second therapeutic agent is pembrolizumab and the treated cancer is a melanoma, lung cancer (e.g., non-small cell lung cancer or small cell lung cancer), Hodgkin's lymphoma, head and neck cancer (e.g., squamous cell carcinoma of the head and neck), primary mediastinal large B-cell lymphoma, urothelial carcinoma, hepatocellular carcinoma, microsatellite instability-high cancer, gastric cancer, esophageal cancer, cervical cancer, Merkel cell carcinoma, kidney carcinoma, or endometrial carcinoma. In some embodiments, the second therapeutic agent is MPDL3280A and the treated cancer is a lung cancer (e.g., non-small cell lung cancer or small cell lung cancer), urothelial carcinoma, hepatocellular carcinoma, or breast cancer. In some embodiments, the second therapeutic agent is MEDI4736 and the treated cancer is a lung cancer (e.g., non-small cell lung cancer or small cell lung cancer) or urothelial carcinoma. In some embodiments, the second therapeutic agent is MSB0010718C and the treated cancer is a urothelial carcinoma. In some embodiments, the second therapeutic agent is MSB0010718C and the treated cancer is a melanoma, lung cancer (e.g., non-small cell lung cancer), colorectal cancer, kidney cancer, ovarian cancer, pancreatic cancer, gastric cancer, and breast cancer.

Advantageously, the compounds of the disclosure and a checkpoint inhibitor may act synergistically to treat cancer. Without wishing to be bound by theory, the synergy between the compounds of the disclosure and checkpoint inhibitors may be attributed to the checkpoint inhibitor efficacy enhancement associated with the BRD9 inhibition-induced downregulation of Foxp3+ Treg cells.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the present disclosure or pharmaceutically acceptable salts thereof are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the disclosure, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. In particular embodiments, a compound of the present disclosure or a pharmaceutically acceptable salt thereof is formulated for intravenous administration.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerin. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the compounds of the disclosure may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Kits

The present disclosure also features kits including (a) a pharmaceutical composition including an agent that reduces the level and/or activity of BRD9 in a cell or subject described herein, and (b) a package insert with instructions to perform any of the methods described herein. In some embodiments, the kit includes (a) a pharmaceutical composition including an agent that reduces the level and/or activity of BRD9 in a cell or subject described herein, (b) an additional therapeutic agent (e.g., an anti-cancer agent), and (c) a package insert with instructions to perform any of the methods described herein.

### EXAMPLES

### Example 1. Flow Cytometry of Peripheral Blood Mononuclear Cells (PBMCs) from Human Synovial Sarcoma Subjects Treated with Compound A Reveals a Decrease in BRD9

Procedure: Serial blood draws from subjects treated with Compound A were collected in sodium heparin tubes at timepoints shown in Table 3. PBMCs were isolated by Ficoll density gradient centrifugation and cryopreserved in Cryostor CS5 (StemCell Technologies). Cryopreserved PBMC aliquots were thawed at 37° C., stained with DyLight 800 maleimide (Thermo Fisher) to label dead cells, blocked with Human Fc block (Biolegend), and incubated with a surface antibody cocktail (see Table 4, antibody clone column) to label specified surface antigens (see Table 4, antigen column). Following surface labeling, cells were fixed and permeabilized using the eBioscience Foxp3/Transcription Factor Staining Buffer Set (Invitrogen) and incubated with an intracellular antibody cocktail to label specified intracellular antigens. Finally, BRD9 was detected using a phycoerythrin (PE)-conjugated anti-rabbit secondary antibody (Cell Signaling Technology), and cells were analyzed by flow cytometry using an LSR Fortessa (BD Biosciences). Population gates were drawn using FlowJo v10.7 software (BD Biosciences) and BRD9 levels were measured as the mean PE fluorescence intensity (MFI) of all cells in each population of interest.

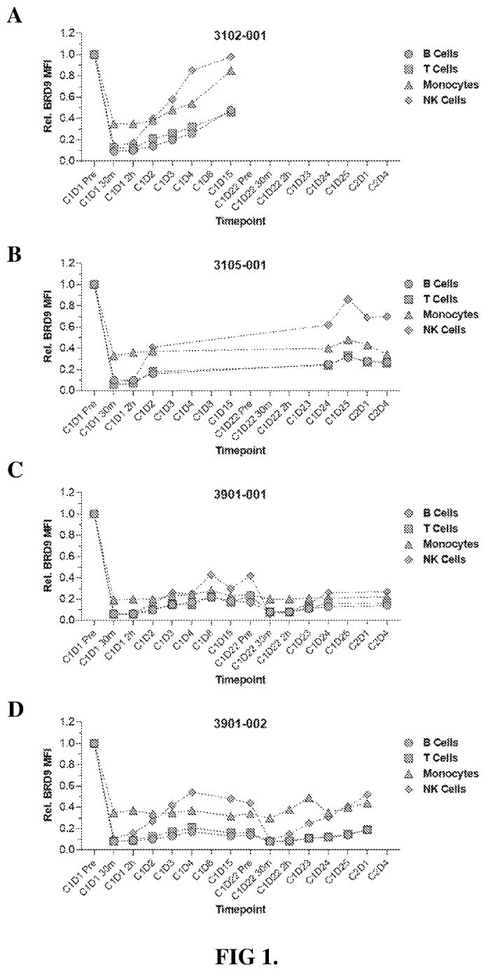

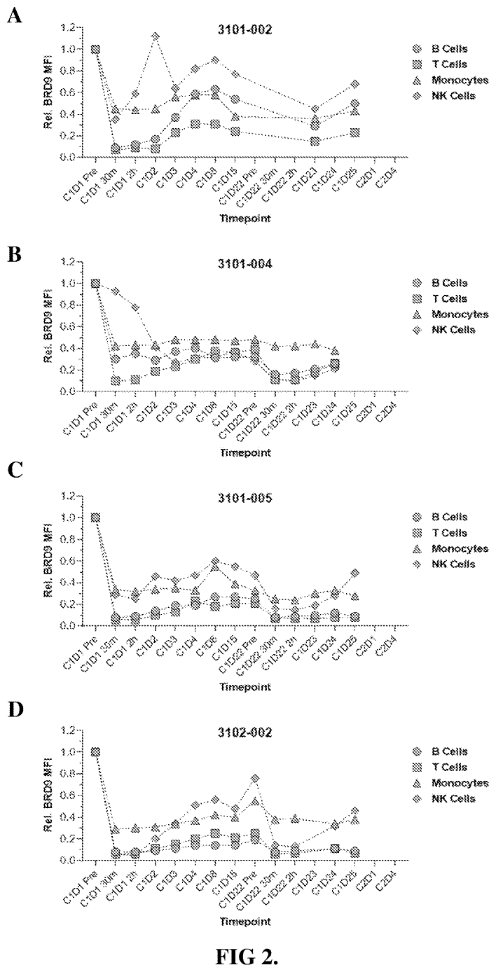

Results: Treatment with Compound A led to a decrease in BRD9 in PBMCs in each of the four subjects dosed at both 5 mg (FIGS. 1A-1D) and 10 mg (FIGS. 2A-2D) dose levels. B cells and T cells showed greater and more sustained reductions in BRD9 than monocytes and natural killer (NK) cells.

TABLE 3
Dosing and blood collection schedule

| Visit | Dosing Day | Blood Timepoint | |
|---|---|---|---|
| C1D1 | X | Pre-dose | |
| | | 30 | min |
| | | 2 | hrs |
| C1D2 | | 22 | hrs |
| C1D3 | | 46 | hrs |
| C1D4 | X | Pre-dose | |
| C1D8 | X | Pre-dose | |
| C1D11 | X | None | |
| C1D15 | X | Pre-dose | |
| C1D18 | X | None | |
| C1D22 | X | Pre-dose | |
| | | 30 | min |
| | | 2 | hrs |
| C1D23 | | 22 | hrs |
| C1D24 | | 46 | hrs |

| | | |
|---|---|---|
| C1D25 | X | Pre-dose |
| C2D1 | X | Pre-dose |
| C2D4 | X | Pre-dose |

C = cycle
D = day

TABLE 4
Flow cytometry panel

| | | | |
|---|---|---|---|
| Antigen | Antibody clone | Fluorophore | Relevant population |

Surface antibody cocktail

| | | | |
|---|---|---|---|
| CD3 | UCHT1 | BV421 | T cells |
| CD4 | RPA-T4 | BV605 | T cells |
| CD8 | SK1 | PerCP-Cy5.5 | T cells |
| CD45 | 2D1 | AF488 | Pan leukocyte |
| CD19 | HIB19 | BV510 | B cells |
| CD14 | 63D3 | AF700 | Monocytes |
| CD16 | 3G8 | PE-Dazzle594 | NK cells |
| CD56 | HCD56 | PE-Cy7 | NK cells |
| CD25 | 2A3 | BUV737 | Tregs |
| CD127 | A019D5 | PE-Cy5 | Tregs |

Intracellular antibody cocktail

| | | | |
|---|---|---|---|
| FoxP3 | 259D | AF647 | Tregs |
| BRD9 | E4Q3F | None | All |

FIGS. 1A-1D are graphs illustrating BRD9 protein levels detected by flow cytometry in PBMC subpopulations from 4 subjects treated with 5 mg Compound A. (A) is subject 1, (B) is subject 2, (C) is subject 3, (D) is subject 4.

FIGS. 2A-4D are graphs illustrating BRD9 protein levels detected by flow cytometry in PBMC subpopulations from 4 subjects treated with 10 mg Compound A. (A) is subject 5, (B) is subject 6, (C) is subject 7, (D) is subject 8.

### Example 2. Immunohistochemistry (IHC) of Tumor Biopsies from Human Synovial Sarcoma Subjects Treated with Compound A Reveals a Reduction in BRD9

Procedure: Screening and on-treatment synovial sarcoma tumor biopsies from subjects treated with Compound A were collected as described in Table 5. Samples were routinely processed for histology using formalin fixation and paraffin embedding, and 4 μm-thick sections were mounted on glass slides for IHC. BRD9 protein was detected using an anti-BRD9 rabbit monoclonal antibody (clone E4Q3F, Cell Signaling Technology), and Ki67 protein was detected using an anti-Ki67 mouse monoclonal antibody (clone K2, Leica Biosystems). IHC was carried out on a Leica Bond RX autostainer, using the BOND Polymer Refine Detection kit with DAB chromogen and hematoxylin counterstain. Slides were reviewed by an MD pathologist and BRD9 levels were reported as an H-score, using the following formula:

$$( 1 \times {\%}_{weak} ) + ( 2 \times {\%}_{moderate} ) + ( 3 \times {\%}_{strong} ) .$$

### Where:

%_{weak}=The percentage of tumor cell nuclei weakly positive for BRD9
%_{moderate}=The percentage of tumor cell nuclei moderately positive for BRD9
%_{strong}=The percentage of tumor cell nuclei strongly positive for BRD9
Ki67 levels were reported as % positive tumor cells.

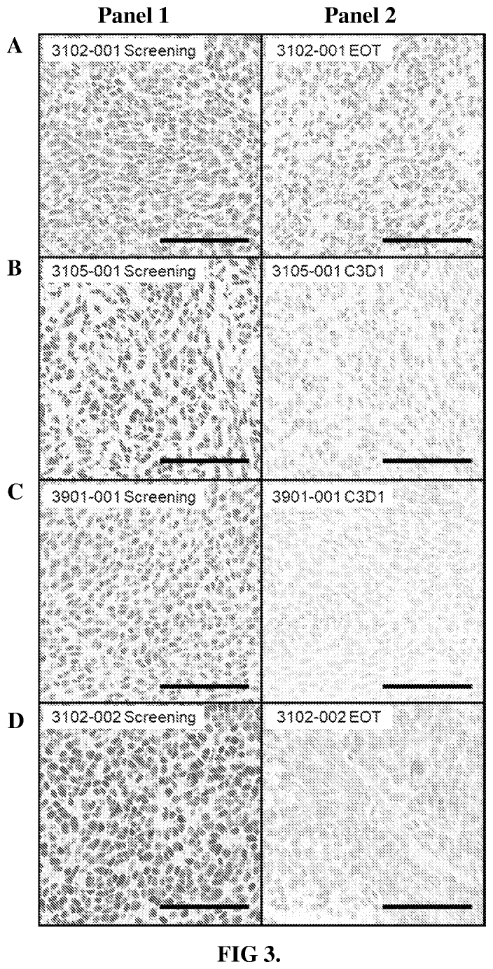

Results: BRD9 was detected in 100% of tumor cells in all screening samples, with the majority of cells exhibiting moderate to strong positivity (Table 5, FIG. 3). BRD9 levels were reduced in all samples that were treated with Compound A, with the greatest reduction occurring in a total weekly dose of 80 mg (40 mg BIW or 80 mg QW) and above (Table 5, FIG. 3). Ki67 levels were generally reduced in samples that had the greatest reduction in BRD9. Thus in 5 out of 5 on-treatment samples with BRD9 H-scores of 0, the percentage of tumor cells positive for Ki67 was reduced by over 50%.

TABLE 5
Biopsy collection information, BRD9 H-Scores and Ki67% change

| | | | | | | Ki67% |
|---|---|---|---|---|---|---|
| | | | | Days | BRD9 H- | Change |
| | | | # Doses | Post- | Score | from |
| Cohort | Subject | Collection | Received | Dose | (0-300) | Screening |
| 5 mg | 1 | Screening | N/A | N/A | 195 | N/A |
| BIW | | EOT | 2 | 13 | 111 | −58% |
| | 2 | Screening | N/A | N/A | 230 | N/A |
| | | C3D1 | 17 | 2 | 85 | +48% |
| | 3 | Screening | N/A | N/A | 228 | N/A |
| | | C3D1 | 17 | 1 | 64 | +67% |
| 10 mg | 4 | Screening | N/A | N/A | 264 | N/A |
| BIW | | EOT | 14 | 4 | 14 | −58% |
| 20 mg | 5 | Screening | N/A | N/A | 225 | N/A |
| BIW | | EOT | 16 | 2 | 61 | −43% |
| | 6 | Screening | N/A | N/A | 202 | N/A |
| | | EOT | 13 | 11 | 6 | −35% |
| 40 mg | 7 | Screening | N/A | N/A | 265 | N/A |
| BIW | | C2D15 | 13 | 1 | 0 | −59% |
| | 8 | Screening | N/A | N/A | 185 | N/A |
| | | C3D1 | 17 | 1 | 0 | −67% |
| | 9* | Screening | N/A | N/A | N/A | N/A |
| | | EOT | 8 | 8 | 12 | N/A |
| 60 mg | 10 | Screening | N/A | N/A | 242 | N/A |
| BIW | | C2D15 | 13 | 1 | 0 | −80% |
| 80 mg | 11 | Screening | N/A | N/A | 185 | N/A |
| BIW | | C3D1 | 17 | 2 | 0 | −68% |
| 80 mg | 12 | Screening | N/A | N/A | 210 | N/A |
| QW | | C3D1 | 7 | 15 | 82 | +39% |
| | 13 | Screening | N/A | N/A | 225 | N/A |
| | | EOT | 8 | 9 | 0 | −66% |
| 120 mg | 14 | Screening | N/A | N/A | 104 | N/A |
| QW | | C2D15 | 7 | 1 | 7 | +67% |

Screening = Screening visit, before treatment began
EOT = End of treatment
C3D1 = Cycle 3, day 1
C2D15 = Cycle 2, day 15
BIW = Twice-weekly dosing
QW = Once-weekly dosing
*screening biopsy was taken but did not have any viable tissue

FIGS. 3A-D show representative images of BRD9 detected by IHC in synovial sarcoma patient tumor biopsies collected at screening and on-treatment visits. (A) is subject 1 screening (Panel 1) and EOT (Panel 2), (B) is subject 2 screening (Panel 1) and C3D1 (Panel 2), (C) is subject 3 screening (Panel 1) and C3D1 (Panel 2), (D) is subject 4 screening (Panel 1) and EOT (Panel 2).

### Example 3. IHC of SYO-1 Xenografts from Mice Treated with Compound A Reveals Dose and Time-Dependent Decreases in BRD9

Procedure: Female BALB/c nude mice were implanted on the right flanks with 5×10⁶ SYO-1 cells per mouse. Treatment, as described in Table 6, began when tumors reached a volume of 66-172 mm³. When endpoints were reached, animals were sacrificed, and tumors were routinely processed for histology using formalin fixation and paraffin embedding. 4 μm-thick sections were mounted on glass slides, and BRD9 protein was detected by immunohistochemistry (IHC), using an anti-BRD9 rabbit monoclonal antibody (clone E4Q3F, Cell Signaling Technology). IHC was carried out on a Leica Bond RX autostainer, using the BOND Polymer Refine Detection kit with DAB chromogen and hematoxylin counterstain. Whole slide image acquisition was performed with an Aperio AT2 scanner (Leica Biosystems), at 20× magnification. HALO image analysis software (Indica Labs) was used to select regions of viable tumor for analysis, and the Indica Labs Multiplex IHC algorithm was used to quantify BRD9 staining intensity on a per-cell basis. H-scores were derived using the following formula:

$$( 1 \times {\%}_{weak} ) + ( 2 \times {\%}_{moderate} ) + ( 3 \times {\%}_{strong} ) .$$

### Where:

%_{weak}=The percentage of cells weakly positive for BRD9
%_{moderate}=The percentage of cells moderately positive for BRD9
%_{strong}=The percentage of cells strongly positive for BRD9

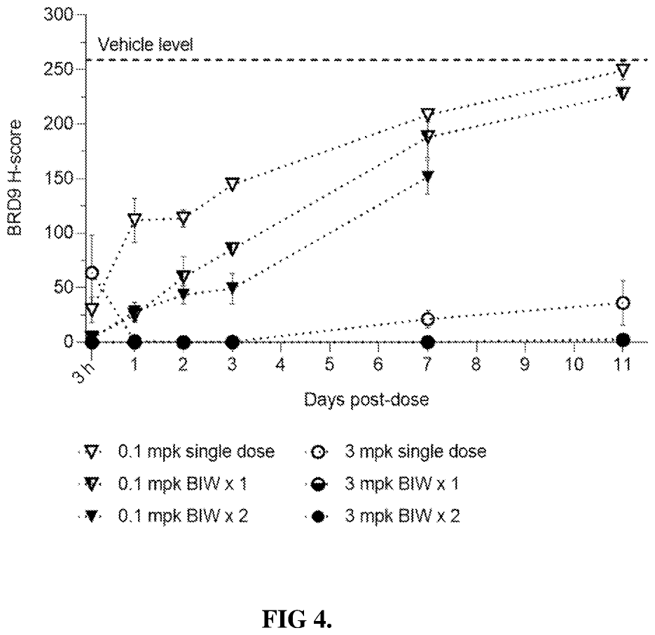

Results: BRD9 was detected in >99% of tumor cells in the xenografts from vehicle-treated animals, with the majority of cells exhibiting moderate to strong positivity. Treatment with either 0.1 mg/kg or 3 mg/kg Compound A led to robust reduction in BRD9 within 3 hours after a single dose. Following the single 0.1 mg/kg dose, BRD9 levels took approximately 11 days to return to baseline. This recovery was slightly delayed following repeat dosing at 0.1 mg/kg. Following a single 3 mg/kg dose, modest recovery of BRD9 was observed after 7 and 11 days. Following repeat dosing at 3 mg/kg, only minimal recovery was observed after 11 days (FIG. 4).

TABLE 6
Study design

| | | | Dose | Dose | Total doses | Timepoints (hours |
|---|---|---|---|---|---|---|
| Group | N | Treatment | (mg/kg) | Schedule | received* | post last dose) |
| 1 | 3 | Vehicle | NA | Single | 1 | 3 |
| 2 | 3 | Vehicle | NA | BIW × 1 | 3 | 3 |
| 3 | 3 | Vehicle | NA | BIW × 2 | 5 | 3 |
| 4 | 18 | Compound A | 0.1 | Single | 1 | 3, 24, 48, 72, 168, 264 |
| 5 | 18 | Compound A | 0.1 | BIW × 1 | 3 | 3, 24, 48, 72, 168, 264 |
| 6 | 15 | Compound A | 0.1 | BIW × 2 | 5 | 3, 24, 48, 72, 168 |
| 7 | 18 | Compound A | 3 | Single | 1 | 3, 24, 48, 72, 168, 264 |
| 8 | 18 | Compound A | 3 | BIW × 1 | 3 | 3, 24, 48, 72, 168, 264 |
| 9 | 18 | Compound A | 3 | BIW × 2 | 5 | 3, 24, 48, 72, 168, 264 |

BIW × 1 = Dosed on days 1, 4 and 8
BIW × 2 = Dosed on days 1, 4, 8, 11 and 15

FIG. 4 is a graph illustrating BRD9 levels detected by IHC in SYO-1 xenografts from mice treated with Compound A. Symbols represent group averages, and error bars represent SEM.

### Example 4: Nanosuspension and Microsuspension Formulations of Compound A

Generally, nanosuspension formulations of Compound A (e.g., 5 mg/ml to 150 mg/ml concentration range) of suitable particle size distribution (e.g., D50 ranging from 1 micron to <500 nm) were prepared using a low energy jar mill (e.g., at 250 rpm or higher for an optimum period of time such as 4 hours) or a high energy mill (e.g., at 1000 rpm or higher for 15-90 minutes) using polymeric or zirconium dioxide beads of 0.3 mm-1 mm size. Suspensions were also obtained by jet milling the drug substance to suitable particle size and using an appropriate vehicle such as low viscosity CMC Na for suspending.

More specifically, nanosuspensions were prepared using two different methods as described below.

### Nanosuspension Preparation Method 1:

Milling was performed in a high energy ball mill with suitable sized-spherical, polymeric or Zirconium oxide beads. Compound A and excipient(s) as indicated in Table 7 were combined in deionized water per the indicated batch size and transferred to the milling chamber. Nanosuspensions A through D were generated utilizing process conditions denoted in Table 8.

### Nanosuspension Preparation Method 2:

Compound A was first mechanically jet-milled to reduce initial particle size prior to ball milling. The jet mill feed pressure was 4.5 bar and milling pressure was 5.0 bar. The resulting particle size of jet milled Compound A was 5-6 microns (D50). Jet-milled Compound A was combined with an aqueous solution of excipient(s) as described in Table 7 and combined with ball milling media (e.g., 0.3 mm diameter ZrO2 beads) to prepare for ball milling. A High-Capacity Laboratory jar mill was used to create nanosuspensions E through G using the conditions listed in Table 8. Formulation EF1 is a dilution of the 40 mg/mL formulation of E or F. Solid state of each formulation was crystalline via XRPD.

TABLE 7
Nanosuspension Formulation Compositions

| | Batch Size | Compound A | | Excipient 2 |
|---|---|---|---|---|
| Formulation | (g) | (wt %) | Excipient 1 (wt %) | (wt %) |
| A | 6.0 | 20 | 2 wt % TWEEN ®20 | — |
| B | 6.2 | 10 | 1.5 wt % TWEEN ®20 | — |
| C | 6.2 | 15 | 2.25 wt % TWEEN ®20 | — |
| D | 6.2 | 10 | 1 wt % TWEEN ®20 | 0.5 wt % poly |
| | | | | (vinyl |
| | | | | pyrrolidone) |
| E | 35 | 4.4 | 0.5 wt % CMC Na1 | 0.2 wt % |
| | | | | TWEEN ®20 |
| F | 17 | 4.2 | 0.5 wt % CMC Na | 0.2 wt % |
| | | | | TWEEN ®20 |
| G | 15 | 10.8 | 0.5 wt % CMC Na | 0.2 wt % |
| | | | | TWEEN ®20 |
| EF1 | | 0.4-0.5% | 0.5 wt % CMC Na | 0.2 wt % |
| | | | | TWEEN ®20 |

¹CMC Na = sodium carboxymethyl cellulose

TABLE 8
Example Nanosuspension Formulation Milling Parameters

| | Preparation | Milling | Mill | Milling | | Milling |
|---|---|---|---|---|---|---|
| | Method | Media, | Speed, | Time | | Temperature |
| Formulation | Used | (vol %) | (RPM) | (min) | | (C) |
| A | 1 | 851 | 2500 | 30 | min | 5º C. |
| B | 1 | 651 | 2500 | 40 | min | 5º C. |
| C | 1 | 651 | 2500 | 40 | min | 5º C. |
| D | 1 | 651 | 2500 | 25 | min | º C. |
| E | 2 | 1.52 | 300 | 13 | hr | RT3 |
| F | 2 | 1.52 | 300 | 15 | hr | RT |
| G | 2 | 1.52 | 300 | 22 | hr | RT |

¹Calculated as volume of the milling chamber.
²Calculated as multiplier of batch size.
³Room Temperature

Other processing conditions and milling media, not represented here, can also be implemented for generating nanosuspensions of Compound A.

Nanosuspensions were characterized with respect to particle size, Compound A concentration, purity, and XRPD (X-Ray Powder Diffraction) (Table 9). As demonstrated by particle size analysis, the milling processes were effective in generating nanoparticles, with a majority of particles measuring at less than 1 micron. Concentrations of Compound A measuring from approximately 4 mg/mL to about 200 mg/mL were achieved.

TABLE 9
Nanosuspension Formulation Characterization Results

| | Particle | Compound A | |
|---|---|---|---|
| | Size D(50), | Concentration, | |
| Formulation | (microns) | (mg/mL) | XRPD |
| A | 0.138 | 200 | Crystalline |
| B | 0.131 | 100 | peaks |
| C | 0.135 | 150 | observed, |
| D | 0.135 | 100 | no form |
| E | 0.273 | 39.2 | change |
| F | 0.309 | 36.1 | |
| G | 0.450 | 91.0 | |
| EF1 | 0.302 | 3.7 mg/ml | |

A microsuspension of jet-milled drug-substance of Compound A was also prepared (Formulation H). Approximately 3 g of Compound A was subjected to a jet mill using a feed pressure of 6 bar and milling pressure of 5 bar. The feed rate was 1.52 g/10 min at this scale. The particle size of formulation H was D50=3.48 microns and D90=8.47 microns. Solid state of the formulation was crystalline via XRPD. The milled material showed comparable physical and chemical properties to the original form of Compound A, including solid form, purity, water uptake.

### Nanosuspension and Microsuspension Rat PK Study

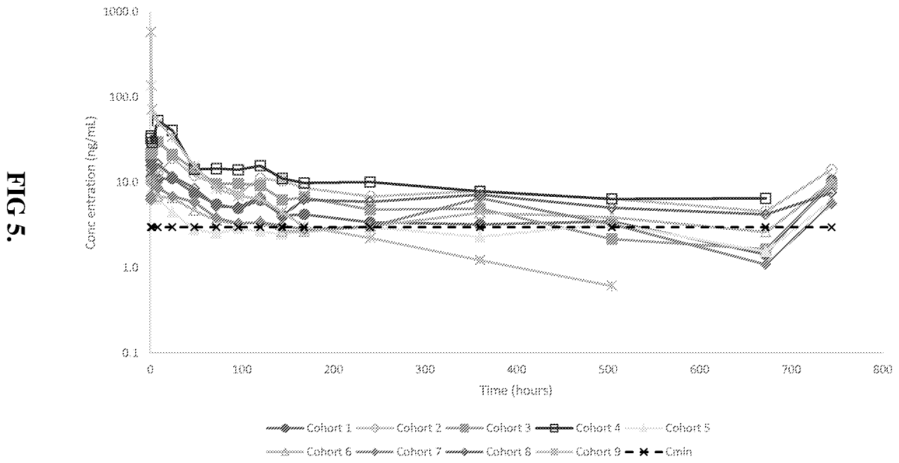

Performance of nanosuspensions E/F and G, and microsuspension H were evaluated in Male SD rats. Note that formulations E and F were used interchangeably in this experiment as they had almost identical properties (see Tables 7-9), which is denoted herein as “E/F”. The animals were approximately 6-8 weeks of age and approximately 200-300 g at time of dosing. An IV formulation (denoted as Formulation IV) was used as a control (20% SBE-CD in 5 mM citrate buffer pH3, final pH adjusted to a pH of 5 by using 1 N NaOH). The nanosuspensions were dosed at 20 mg/kg and 40 mg/kg and the microsuspension was dosed at 40 mg/kg in saline (Table 10). Both subcutaneous (SC) and intramuscular (IM) routes of administration were evaluated. At each time point, approximately 0.2 mL blood was collected via jugular vein and analyzed by LC-MS/MS for mean plasma concentration of Compound A (FIG. 5). Pharmacokinetics determined that a target Cmin of 3 ng/mL was desired, which is denoted by the black dashed line in FIG. 5.

TABLE 10
Rat PK Study Parameters of Nanosuspension
and Microsuspension Formulations

| | | | Initial | | |
|---|---|---|---|---|---|
| | Legend | | concentration | Dose | |
| | in | | of Formulation | administered | Route of |
| Cohort | FIG. 5 | Formulation | (mg/mL) | (mg/kg) | administration |
| 1 | • | E/F | 40 | 20 | SC |
| 2 | ○ | E/F | 40 | 40 | SC |
| 3 | ▪ | E/F | 40 | 20 | IM |
| 4 | □ | G | 100 | 40 | IM |
| 5 | ▴ | H | 40 | 20 | SC |
| 6 | Δ | H | 40 | 40 | SC |
| 7 | ♦ | H | 40 | 20 | IM |
| 8 | ⋄ | H | 40 | 40 | IM |
| 9 | X | IV | — | — | IV |

FIG. 5 shows that nanosuspensions E-G and microsuspension H met the target Cmin criteria of 3 ng/mL over a course of two weeks (336 hours). Further, at the same dose level, IM and SC routes of administration of these formulations were comparable at two weeks, and exposure is dose proportional at terminal time-point(s). At a dose of 40 mg/kg, nanosuspensions E-G and microsuspension H met Cmin criteria of 3 ng/mL for at least 4 weeks (672 hours). These data suggest that nanosuspension and microsuspension formulations of Compound A are a good format for long acting or sustained drug release, and for maintaining a target plasma concentration of 3 ng/mL over a dosing cycle.

### Nanosuspension Mouse PK/PD Study

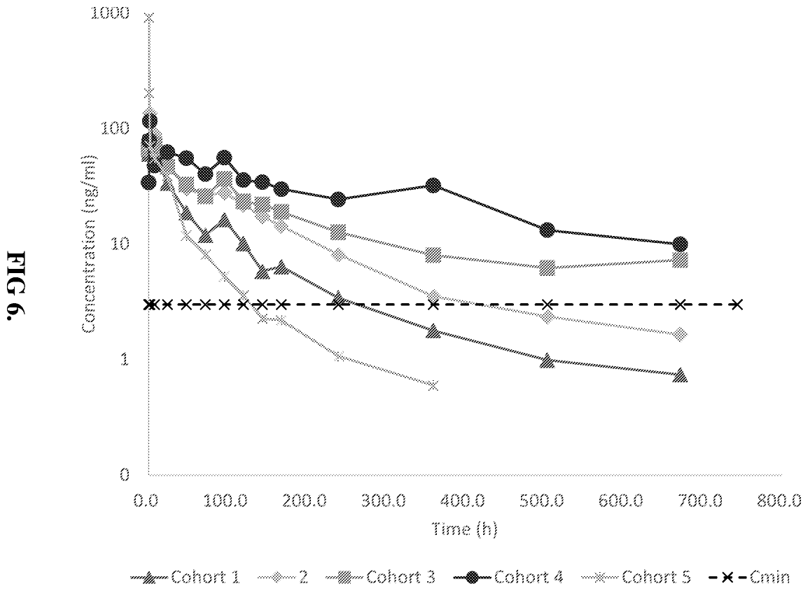

Performance of nanosuspensions E/F and EF1 were evaluated in Male CD1 mice. The animals were approximately 6-8 weeks of age and weighed approximately 20-30 g at dosing. The nanosuspensions were dosed sub-cutaneously at 8 mg/kg, 30 mg/kg. 90 mg/kg and 200 mg/kg (Table 11). At each time point, approximately 0.03 mL blood was collected via jugular vein and analyzed by LC-MS/MS for mean plasma concentration of Compound A (FIG. 6). Pharmacokinetics determined that a target Cmin of 3 ng/mL was desired, which is denoted by the black dashed line in FIG. 6.

TABLE 11
Mouse PK Study Parameters of Nanosuspension Formulations

| | | | Initial | | |
|---|---|---|---|---|---|
| | | | concentration | Dose | |
| | Legend in | | of Formulation | administered | Route of |
| Cohort | FIG. 6 | Formulation | (mg/mL) | (mg/kg) | administration |
| 1 | ▴ | EF1 | 5 | 8 | SC |
| | | | (diluted from | | |
| | | | 40 mg/mL | | |
| | | | formulation) | | |
| 2 | ♦ | EF1 | 5 | 30 | SC |
| | | | (diluted from | | |
| | | | 40 mg/mL | | |
| | | | formulation) | | |
| 3 | ▪ | E/F | 40 | 90 | SC |
| 4 | • | E/F | 40 | 200 | SC |
| 5 | * | IV | — | 8 | IV |

FIG. 6 shows that nanosuspensions E and/or F administered at a dose of 30 mg/kg or higher met the target Cmin criteria of 3 ng/ml over a course of two weeks (336 hours). At a dose of 90 mg/kg or higher, mg/mL, nanosuspensions E and/or F met Cmin criteria of 3 ng/ml for at least 4 weeks (672 hours). Further, exposure is dose proportional at 2 weeks. These data suggest that nanosuspension formulations of Compound A are a good format for long acting or sustained drug release, and for maintaining a target plasma concentration of 3 ng/mL over a dosing cycle.

### Nanosuspension Mouse Efficacy and PK/PD Study

### Efficacy:

Each mouse was inoculated subcutaneously on the right flank with a single cell suspension of SYO-1 human biphasic synovial sarcoma tumor cells (5×10⁶) in 100 μL DMEM with 10% FBS for the tumor development. Treatment (Table 12) was started when mean tumor volume reached about 100-150 mm³ for the efficacy cohorts. Based on the tumor volume, mice were randomly assigned to respective groups using a computer-generated randomization procedure. Corresponding formulations are in Table 12. For each dosing cohort, dosing volume was calculated based on concentration of the formulation and dose volume (ml/kg). Vehicle for IV was 20% HP-β-CD in 5 mM citrate buffer of pH 3, with adjustment to a final PH of 5. Body weight of all animals was measured and recorded twice a week throughout the study. The measurement of tumor size was conducted twice a week with a caliper and recorded. Tumor volume was used for calculation of tumor growth inhibition (TGI) value of the tumors in the treated and the control cohorts, respectively, on a given day after tumor inoculation.

64 plasma samples were collected at two time points (672 h and 676 h) from efficacy study cohorts. 64 tumor samples were collected at two time points (672 h and 676 h) from efficacy cohorts 1-8 and cut into 3 pieces: one piece for PK analysis, one piece for WB analysis (BRD9), and one piece snap frozen for further analysis (n=4). Animals that were observed to be in a continuing deteriorating condition or their tumor size exceeding 2,000 mm³ were euthanized prior to death, or before reaching a comatose state. The entire cohort was terminated when the mean tumor exceeded 2000 mm³ in that cohort.

TABLE 12
Groups and Treatment for Mouse Efficacy Experiment

| | | # | | | Formulation | | | | Collection |
|---|---|---|---|---|---|---|---|---|---|
| | | Animals/ | Dose | Vol | & Initial | | Dosing | Study | time points |
| Cohort | Drug | cohort | (mg/kg) | (ml/kg) | Conc. (mg/mL) | Route | Freq. | Duration | (hr) |
| 1 | Vehicle\hat{ } | 8 | — | 5 | Vehicle\hat{ } | IV | BIW | 4 | 672 |
| | | | | | | | | weeks | |
| 2 | Compound | 8 | 1 | 5 | 0.2 mg/mL | IV | BIW | 4 | 672 |
| | A | | | | of IV* | | | weeks | |
| 3 | Compound | 8 | 1 + 30 | 5 + 6 | 0.2 mg/mL | IV + | 1 dose + | 4 | 672 |
| | A | | | | of IV* + | SC | 1 dose | weeks | |
| | | | | | 5 mg/mL of | | | | |
| | | | | | Formulation | | | | |
| | | | | | EF1 (diluted | | | | |
| | | | | | from 40 mg/mL | | | | |
| | | | | | formulation) | | | | |
| 4 | Compound | 8 | 200 | 5 | 40 mg/ml of | SC | 1 dose | 4 | 672 |
| | A | | | | Formulation | | | weeks | |
| | | | | | E/F | | | | |
| 5 | Compound | 8 | 30 | 6 | 5 mg/mL of | SC | 1 dose | 4 | 672 |
| | A | | | | Formulation | | | weeks | |
| | | | | | E/F (diluted | | | | |
| | | | | | from 40 mg/mL | | | | |
| | | | | | formulation) | | | | |
| 6 | Compound | 8 | 8 | 1.6 | 5 mg/mL of | SC | 1 dose | 4 | 672 |
| | | | | | Formulation | | | weeks | |
| | A | | | | E/F (diluted | | | | |
| | | | | | from 40 mg/mL | | | | |
| | | | | | formulation) | | | | |
| 7 | Compound | 8 | 200 | 2 | 100 mg/ml of | IM | 1 dose | 4 | 672 |
| | A | | | | Formulation G | | | weeks | |
| 8 | Compound | 8 | 8 | 1.6 | 5 mg/mL of | IM | 1 dose | 4 | 672 |
| | A | | | | Formulation | | | weeks | |
| | | | | | EF1 (diluted | | | | |
| | | | | | from 40 mg/mL | | | | |
| | | | | | formulation) | | | | |

\hat{ }Vehicle for IV was 20% HP-β-CD in 5 mM citrate buffer of pH 3, with adjustment to a final PH of 5.
*IV formulation is 20% SBE-CD in 5 mM citrate buffer pH 3 final pH to 5 adjusted by 1N NaOH

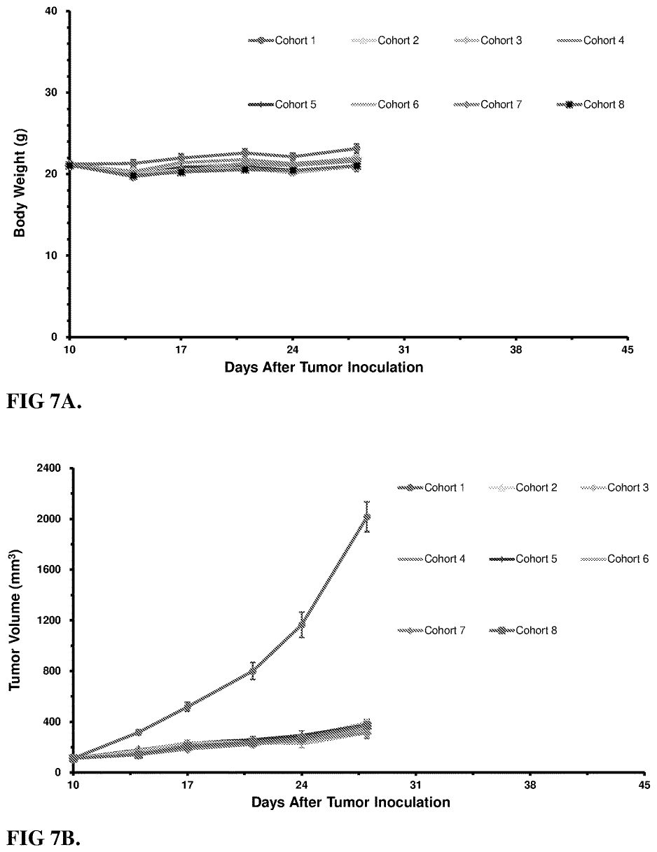

FIG. 7A shows body weight and FIG. 7B shows tumor volume results for the duration of the efficacy experiment. Animals tolerated all doses and all modes of administration similarly (FIG. 7A) with body weight remaining fairly constant throughout the experiment. Further, all doses and all modes of administration (Cohorts 2-8) slowed tumor growth significantly over 4 weeks in comparison to the Vehicle control (Cohort 1) (FIG. 7B). These data suggest that nanosuspension formulations of Compound A are a good format for long acting or sustained drug release, and that IV, SC, and IM modes of administration are effective. Further, these data suggest that nanosuspension formulations of Compound A are capable of maintaining a target plasma concentration of 3 ng/mL over a dosing cycle.

### PK/PD:

Treatment (Table 13) starts when mean tumor volume reaches about 300-400 mm³ for the PKPD cohorts. Based on the tumor volume, mice are randomly assigned to respective groups using a computer-generated randomization procedure. Corresponding formulations are shown in Table 13. For each dosing cohort, dosing volume is calculated based on concentration of the formulation and dose volume (ml/kg). Vehicle for IV is 20% HP-β-CD in 5 mM citrate buffer of pH 3, with adjustment to a final PH of 5. Body weight of all animals is measured and recorded twice a week throughout the study. The measurement of tumor size is conducted twice a week with a caliper and recorded. Tumor volume is used for calculation of tumor growth inhibition (TGI) value of the tumors in the treated and the control cohorts, respectively, on a given day after tumor inoculation.

111 plasma samples are collected at five time points (4 h, 24 h, 96 h, 168 h and 336 h) from PK/PD cohorts 9-16 for PK analysis (n=3). 111 tumor samples are collected at five time points (4 h, 24 h, 96 h, 168 h and 336 h) post last dose from PK/PD cohorts 9-16 and cut into 3 pieces: one piece for PK analysis, one piece for WB analysis (BRD9), and one piece snap frozen for further analysis (n=3). Animals observed to be in a continuing deteriorating condition or their tumor size exceeding 2,000 mm³ will be euthanized prior to death, or before reaching a comatose state. An entire cohort will be terminated when the mean tumor exceeded 2000 mm³ in that cohort.

TABLE 13
Groups and Treatment for Mouse PKPD Experiment

| | | # | | | Formulation | | | | Collection |
|---|---|---|---|---|---|---|---|---|---|
| | | Animals/ | Dose | Vol | & Initial | | | Study | time points |
| Cohort | Drug | cohort | (mg/kg) | (ml/kg) | Conc. (mg/mL) | Route | Frequency | Duration | (hr) |
| 9 | Vehicle\hat{ } | 6 | — | 5 | Vehicle\hat{ } | IV | single | 4 | 4, 24, 96, |
| | | | | | | | dose | weeks | 168 and 336 |
| 10 | Compound | 15 | 1 | 5 | 0.2 mg/mL | IV | single | 4 | 4, 24, 96, |
| | A | | | | of IV* | | dose | weeks | 168 and 336 |
| 11 | Compound | 15 | 1 + 30 | 5 + 6 | 0.2 mg/mL | IV + | single | 4 | 4, 24, 96, |
| | A | | | | of IV* + | SC | dose | weeks | 168 and 336 |
| | | | | | 5 mg/mL of | | | | |
| | | | | | Formulation | | | | |
| | | | | | EF1 (diluted | | | | |
| | | | | | from 40 mg/mL | | | | |
| | | | | | formulation) | | | | |
| 12 | Compound | 15 | 200 | 5 | 40 mg/ml of | SC | single | 4 | 4, 24, 96, |
| | A | | | | Formulation | | dose | weeks | 168 and 336 |
| | | | | | E/F | | | | |
| 13 | Compound | 15 | 30 | 6 | 5 mg/mL of | SC | single | 4 | 4, 24, 96, |
| | A | | | | Formulation | | dose | weeks | 168 and 336 |
| | | | | | EF1 (diluted | | | | |
| | | | | | from 40 mg/mL | | | | |
| | | | | | formulation) | | | | |
| 14 | Compound | 15 | 8 | 1.6 | 5 mg/mL of | SC | single | 4 | 4, 24, 96, |
| | A | | | | Formulation | | dose | weeks | 168 and 336 |
| | | | | | EF1 (diluted | | | | |
| | | | | | from 40 mg/mL | | | | |
| | | | | | formulation) | | | | |
| 15 | Compound | 15 | 200 | 2 | 100 mg/ml of | IM | single | 4 | 4, 24, 96, |
| | A | | | | Formulation | | dose | weeks | 168 and 336 |
| | | | | | G | | | | |
| 16 | Compound | 15 | 8 | 1.6 | 5 mg/mL of | IM | single | −4 | 4, 24, 96, |
| | A | | | | Formulation | | dose | weeks | 168 and 336 |
| | | | | | EF1 (diluted | | | | |
| | | | | | from 40 mg/mL | | | | |
| | | | | | formulation) | | | | |

\hat{ }Vehicle for IV was 20% HP-β-CD in 5 mM citrate buffer of pH 3, with adjustment to a final PH of 5.
*IV formulation is 20% SBE-CD in 5 mM citrate buffer pH 3 final pH to 5 adjusted by 1N NaOH

### Example 5. Hematoxylin and Eosin (H&E) Staining of Tumor Biopsies from Human Synovial Sarcoma Subjects Treated with Compound A Reveals Treatment Effects Associated with BRD9 Degradation

Procedure: Screening and on-treatment synovial sarcoma tumor biopsies from subjects treated with Compound A were collected as described in Table 5. Samples were routinely processed for histology using formalin fixation and paraffin embedding. 4 μm-thick sections were mounted on glass slides and routinely H&E stained. Slides were reviewed by an MD pathologist and assessed for treatment-related histological changes.

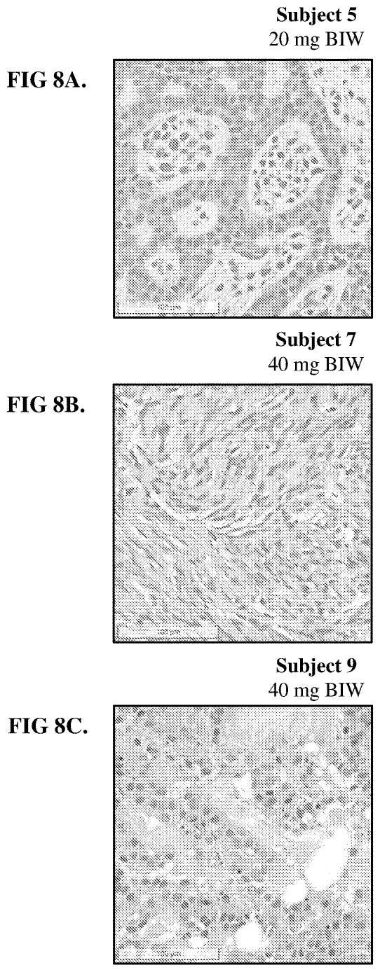

Results: Histological changes were noted at the 20 mg BIW dose and above, but a consistent change was not observed. The Subject 5 sample displayed a highly unusual epithelioid change, with clusters of cells showing more condensed chromatin (FIG. 8A). Increased apoptosis was also observed. Substantial cell dropout with increased extracellular matrix deposition was observed in the Subject 7 sample (FIG. 8B). The Subject 9 sample displayed areas with high numbers of apoptotic cells, as well as possible epithelioid change (FIG. 8C). While these observations were noted by the pathologist as likely to be treatment-related effects, there was not a corresponding screening sample (Table 5) available for comparison.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A method of treating cancer in a subject in need thereof, the method comprising administering an effective amount of a compound that reduces the level and/or activity of BRD9, or a pharmaceutically acceptable salt thereof, wherein the effective amount is an amount sufficient to decrease a BRD9 immunohistochemistry score of the subject.

2. A method of decreasing a BRD9 immunohistochemistry score in a subject, the method comprising administering an effective amount of a compound that reduces the level and/or activity of BRD9, or a pharmaceutically acceptable salt thereof.

3. The method of claim 1, wherein the effective amount is an amount sufficient to decrease a BRD9 immunohistochemistry score in the subject compared to a BRD9 immunohistochemistry score of the subject prior to the administration of the compound or a pharmaceutically acceptable salt thereof.

4. The method of claim 1, wherein the effective amount is an amount sufficient to decrease a BRD9 immunohistochemistry score in the subject compared to a reference BRD9 immunohistochemistry score.

5. The method of claim 1, wherein the effective amount is an amount sufficient to decrease a BRD9 immunohistochemistry score in the subject compared to a BRD9 immunohistochemistry score of a subject who was not administered the compound or a pharmaceutically acceptable salt thereof.

6. The method of claim 1, wherein the BRD9 immunohistochemistry score is an H score.

7. The method of claim 1, wherein the effective amount is an amount sufficient to reduce a BRD9 immunohistochemistry score to a score of between about 15 and about 0.

8-9. (canceled)

10. The method of claim 1, wherein the effective amount is an amount sufficient to maintain a BRD9 immunohistochemistry score that is between about 15 and about 0 in the subject, over at least 14 days.

11-12. (canceled)

13. A method of treating cancer in a subject in need thereof, the method comprising administering an effective amount of a compound that reduces the level and/or activity of BRD9, or a pharmaceutically acceptable salt thereof, wherein the effective amount is an amount sufficient to reduce the level of BRD9 expression in the subject.

14. (canceled)

15. The method of claim 13, wherein the effective amount is an amount sufficient to reduce the level of BRD9 expression in the subject compared to the level of BRD9 expression in the subject prior to the administration of the compound or a pharmaceutically acceptable salt thereof.

16. The method of claim 13, wherein the effective amount is an amount sufficient to reduce the level of BRD9 expression in the subject compared to the level of BRD9 expression of a reference.

17. The method of claim 13, wherein the effective amount is an amount sufficient to reduce the level of BRD9 expression in a subject compared to the level of BRD9 expression in a subject who was not administered the compound or a pharmaceutically acceptable salt thereof.

18-26. (canceled)

27. The method of claim 1, wherein the effective amount is an amount sufficient to cause a greater than or equal to 10% decrease in tumor size and/or a greater than or equal to 15% decrease in tumor attenuation in the subject.

28-29. (canceled)

30. The method of claim 1, wherein the effective amount is an amount sufficient to maintain a plasma concentration of 3 ng/mL of the compound in a subject, over at least 14 days.

31-43. (canceled)

44. The method of claim 1, wherein the cancer is a malignant rhabdoid tumor, a CDS+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, a sarcoma, non-small cell lung cancer, stomach cancer, breast cancer, chordoma, or SMARCAB1-loss cancer.

45-47. (canceled)

48. The method of claim 44, wherein the cancer is a SMARCAB1-loss cancer.

49. The method of claim 48, wherein the SMARCAB1-loss cancer is high grade ovarian serous adenocarcinoma, epithelioid sarcoma, atypical teratoid/rhabdoid tumor, prostate adenocarcinoma, conventional glioblastoma multiforme, breast invasive ductal carcinoma, sarcoma (NOS), gastrointestinal stromal tumor, kidney medullary carcinoma, pancreatic neuroendocrine neoplasm, sinonasal adenocarcinoma, adrenal cortex carcinoma, proximal-type epithelioid sarcoma, head and neck carcinoma, invasive breast carcinoma, rhabdoid tumor of the kidney, dedifferentiated liposarcoma, pancreatic adenocarcinoma, chordoma, testicular yolk sac tumor, small cell lung carcinoma, squamous cell lung carcinoma, acral lentiginous melanoma, peripheral T-cell lymphoma, nasal cavity and paranasal sinus squamous cell carcinoma, malignant peripheral nerve sheath tumor, breast invasive lobular carcinoma, chordoma, a SMARCB1-mutant cancer, or lung adenocarcinoma.

50. (canceled)

51. The method of claim 1, wherein the compound is 3-(6-(7-((1-(4-(6-(azetidin-1-yl)-2-methyl-1-oxo-1,2-dihydro-2,7-naphthyridin-4-yl)-2,6-dimethoxybenzyl)piperidin-4-yl)methyl)-2,7-diazaspiro[3.5]nonan-2-yl)-1-oxoisoindolin-2-yl)piperidine-2,6-dione, or a pharmaceutically acceptable salt thereof.

52. The method of claim 1, wherein the effective amount of the compound or a pharmaceutically acceptable salt thereof, is administered in a dose of 20-80 mg/kg.

53-58. (canceled)

59. The method of claim 1, wherein the effective amount of the compound or a pharmaceutically acceptable salt thereof, is administered to the subject at least once per week.

60-74. (canceled)
